SEQUENCE LISTING (37 sequences)

SEQ ID NO: 1 - protein, 8 aa, Artificial Sequence
  Description of Artificial Sequence Synthetic peptide
SIYRYYGL

SEQ ID NO: 2 - protein, 8 aa, Artificial Sequence
  Description of Artificial Sequence Synthetic peptide
SIINFEKL

SEQ ID NO: 3 - DNA, 63 bp, Artificial Sequence
  Description of Artificial Sequence Synthetic nucleotide primer
atattttctgttattgcttcagttttagcagctagcttggataaaagannsnnsnnsaaattc

SEQ ID NO: 4 - protein, 9 aa, Artificial Sequence
  Description of Artificial Sequence Synthetic peptide
QLSPFPFDL

SEQ ID NO: 5 - protein, 8 aa, Artificial Sequence
  Description of Artificial Sequence Synthetic peptide
EQYKFYSV

SEQ ID NO: 6 - protein, 8 aa, Artificial Sequence
  Description of Artificial Sequence Synthetic peptide
NNNKFYSV

SEQ ID NO: 7 - DNA, 79 bp, Artificial Sequence
  Description of Artificial Sequence Synthetic nucleotide primer
caatggctagcggtggacttaagggtggaccaggtggaggttcaggaggtggaggcccacactcgctgaggtatttcgt

SEQ ID NO: 8 - DNA, 72 bp, Artificial Sequence
  Description of Artificial Sequence Synthetic nucleotide primer
tgaacctccgcctcctgatccaccgccacctgaacctattccaccctcccatctcagggtgaggggctcagg

SEQ ID NO: 9 - DNA, 73 bp, Artificial Sequence
  Description of Artificial Sequence Synthetic nucleotide primer
ggtggaataggttcaggtggcggtggatcaggaggcggaggttcaatccagaaaacccctcaaattcaagtat

SEQ ID NO: 10 - DNA, 71 bp, Artificial Sequence
  Description of Artificial Sequence Synthetic nucleotide primer
gttccctcgagctattacaagtcttcttcagaaataagcttttgttccatgtctcgatcccagtagacggt

SEQ ID NO: 11 - DNA, 27 bp, Artificial Sequence
  Description of Artificial Sequence Synthetic nucleotide primer
tctcaagaattctacttcatacatttt

SEQ ID NO: 12 - DNA, 27 bp, Artificial Sequence
  Description of Artificial Sequence Synthetic nucleotide primer
gtatctgctagctgctaaaactgaagc

SEQ ID NO: 13 - DNA, 72 bp, Artificial Sequence
  Description of Artificial Sequence Synthetic nucleotide primer
atactagctagcttggataaaaggtctatttatagatattatggtttgcttaagggtggaccaggtggaggt

SEQ ID NO: 14 - DNA, 42 bp, Artificial Sequence
  Description of Artificial Sequence Synthetic nucleotide primer
caatccagatctttactaatgcaagtcttcttcagaaataag

SEQ ID NO: 15 - DNA, 30 bp, Artificial Sequence
  Description of Artificial Sequence Synthetic nucleotide primer
ggatatcatatgcaggaactgacaactata

SEQ ID NO: 16 - DNA, 43 bp, Artificial Sequence
  Description of Artificial Sequence Synthetic nucleotide primer
atttgcagatctcgagttactaagcgtagtctggaacgtcgta

SEQ ID NO: 17 - DNA, 42 bp, Artificial Sequence
  Description of Artificial Sequence Synthetic nucleotide primer
ctagcttggataaaaggagcatcatcaattttgaaaagcttc

SEQ ID NO: 18 - DNA, 42 bp, Artificial Sequence
  Description of Artificial Sequence Synthetic nucleotide primer
ttaagaagcttttcaaaattgatgatgctccttttatccaag

SEQ ID NO: 19 - DNA, 42 bp, Artificial Sequence
  Description of Artificial Sequence Synthetic nucleotide primer
ctagcttggataaaagggaacaatacaaattctactcagttc

SEQ ID NO: 20 - DNA, 42 bp, Artificial Sequence
  Description of Artificial Sequence Synthetic nucleotide primer
ttaagaactgagtagaatttgtattgttcccttttatccaag

SEQ ID NO: 21 - DNA, 1632 bp, Artificial Sequence
  Description of Artificial Sequence  Synthetic nucleotide
atgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggctagtggtggtggtggttctggtggtggtggttctggtggtggtggttctgctagcggtggacttaagggtggaccaggtggaggttcaggaggtggaggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctactacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagtggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagggtggaataggttcaggtggcggtggatcaggaggcggaggttcaatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgacagtatggccgagcccaagaccgtctactgggatcgagacatggaacaaaagcttatttctgaagaagacttgtaatagctcgag

SEQ ID NO: 22 - protein, 540 aa, Artificial Sequence
  Description of Artificial Sequence Synthetic peptide
MQLLRCFSIFSVIASVLAQELTTICEQIPSPTLESTPYSLSTTTILANGKAMQGVFEYYKSVTFVSNCGSHPSTTSKGSPINTQYVFKDNSSTIEGRYPYDVPDYALQASGGGGSGGGGSGGGGSASGGLKGGPGGGSGGGGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEGGIGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHDSMAEPKTVYWDRDMEQKLISEEDL

SEQ ID NO: 23 - DNA, 1640 bp, Artificial Sequence
  Description of Artificial Sequence Synthetic nucleotide
gaattctacttcatacattttcaattaagatgcagttacttcgctgtttttcaatattttctgttattgcttcagttttagcagctagcttggataaaagatctatttatagatattatggtttgcttaagggtggaccaggtggaggttcaggaggtggaggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctactacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagtggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagggtggaataggttcaggtggcggtggatcaggaggcggaggttcaatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgacagtatggccgagcccaagaccgtctactgggatcgagacatggaacaaaagcttatttctgaagaagacttgcatatgcaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgcttagtaactcgag

SEQ ID NO: 24 - protein, 542 aa, Artificial Sequence
  Description of Artificial Sequence Synthetic peptide
ILLHTFSIKMQLLRCFSIFSVIASVLAASLDKRSIYRYYGLLKGGPGGGSGGGGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEGGIGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHDSMAEPKTVYWDRDMEQKLISEEDLHMQELTTICEQIPSPTLESTPYSLSTTTILANGKAMQGVFEYYKSVTFVSNCGSHPSTTSKGSPINTQYVFKDNSSTIEGRYPYDVPDYA

SEQ ID NO: 25 - DNA, 1640 bp, Artificial Sequence
  Description of Artificial Sequence Synthetic nucleotide
gaattctacttcatacattttcaattaagatgcagttacttcgctgtttttcaatattttctgttattgcttcagttttagcagctagcttggataaaagagaacaatacaaattctactcagttcttaagggtggaccaggtggaggttcaggaggtggaggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctactacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagtggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagggtggaataggttcaggtggcggtggatcaggaggcggaggttcaatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgacagtatggccgagcccaagaccgtctactgggatcgagacatggaacaaaagcttatttctgaagaagacttgcatatgcaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgcttagtaactcgag

SEQ ID NO: 26 - protein, 542 aa, Artificial Sequence
  Description of Artificial Sequence Synthetic peptide
ILLHTFSIKMQLLRCFSIFSVIASVLAASLDKREQYKFYSVLKGGPGGGSGGGGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEGGIGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHDSMAEPKTVYWDRDMEQKLISEEDLHMQELTTICEQIPSPTLESTPYSLSTTTILANGKAMQGVFEYYKSVTFVSNCGSHPSTTSKGSPINTQYVFKDNSSTIEGRYPYDVPDYA

SEQ ID NO: 27 - DNA, 1640 bp, Artificial Sequence
  Description of Artificial Sequence Synthetic nucleotide
gaattctacttcatacattttcaattaagatgcagttacttcgctgtttttcaatattttctgttattgcttcagttttagcagctagcttggataaaaggagcatcatcaattttgaaaagcttcttaagggtggaccaggtggaggttcaggaggtggaggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctactacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagtggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagggtggaataggttcaggtggcggtggatcaggaggcggaggttcaatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgacagtatggccgagcccaagaccgtctactgggatcgagacatggaacaaaagcttatttctgaagaagacttgcatatgcaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgcttagtaactcgag

SEQ ID NO: 28 - protein, 541 aa, Artificial Sequence
  Description of Artificial Sequence Synthetic peptide
ILLHTFSIKMQLLRCFSIFSVIASVLAASLDKRSIINFEKLLKGGPGGGSGGGGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEGGIGSGGGGSGGGGSIQKTPQIQVYSRHPPENGKPNILNCYVTQFHPPHIEIQMLKNGKKIPKVEMSDMSFSKDWSFYILAHTEFTPETDTYACRVKHDSMAEPKTVYWDRDMEQKLISEEDLHMQELTTICEQIPSPTLESTPYSLSTTTILANGKAMQGVFEYYKSVTFVSNCGSHPSTTSKGSPINTQYVFKDNSSTIEGRYPYDVPDYA

SEQ ID NO: 29 - DNA, 1631 bp, Artificial Sequence
  Description of Artificial Sequence Synthetic nucleotide
atgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggctagtggtggtggtggttcggtggtggtggttctggtggtggtggttctgctagcggtggacttaagggtggaccaggtggaggttcaggaggtggaggcccacactcgatgcggtatttcgagaccgcggtgtcccggcgcggcctcggggagccccggtacatctctgtcggctatgtgaacgacaaggagttcgtgcgcttcgacagcgacgcggagaatccgagatatgagccgagggcgccgtggatggagcaggaggggccggagtattgggagcggatcacgcagatcgccaagggccaggagcagtggttccgagtgaacctgaggaccctgctcggctactacaaccagagcgcgggcggcactcacacactccagtggatgtacggctgtgacgtggggtcggacgggcgcctcctccgcgggtacgagcagttcgcctacgacggctgcgattacatcgccctgaacgaagacctgaaaacgtggacgttcgcggacatgtcgtcgatgatcacccgacgcaagtgggagcaggctggtgctgcagagtattacagggcctacctggagggcgagtgcgtggagtggctccacagatacctgaagaacgggaatgctacgctgctgcgcacagattccccaaaggcacatgtgacctatcaccccagatctaaaggtgaagtcaccctgaggtgctgggccctgggcttctaccctgctgacatcaccctgacctggcagttgaatggggaggagctgacccaggacatggagcttgtggagaccaggcctgcaggggatggaaccttccagaagtgggcatctgtggtggtgcctcttgggaaggagcagaattacacatgccgtgtgtaccatgaggggctgccccatcccctcaccctgagatgggagggtggaataggttcaggtggcggtggatcaggaggcggaggttcaatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgacagtatggccgagcccaagaccgtctactgggatcgagacatggaacaaaagcttatttctgaagaagacttgtaatagctcgag

SEQ ID NO: 30 - protein, 540 aa, Artificial Sequence
  Description of Artificial Sequence Synthetic peptide
MQLLRCFSIFSVIASVLAQELTTICEQIPSPTLESTPYSLSTTTILANGKAMQGVFEYYKSVTFVSNCGSHPSTTSKGSPINTQYVFKDNSSTIEGRYPYDVPDYALQASGGGGSGGGGSGGGGSASGGLKGGPGGGSGGGGPHSMRYFETAVSRRGLGEPRYISVGYVNDKEFVRFDSDAENPRYEPRAPWMEQEGPEYWERITQIAKGQEQWFRVNLRTLLGYYNQSAGGTHTLQWMYGCDVGSDGRLLRGYEQFAYDGCDYIALNEDLKTWTFADMSSMITRRKWEQAGAAEYYRAYLEGECVEWLHRYLKNGNATLLRTDSPKAHVTYHPRSKGEVTLRCWALGFYPADITLTWQLNGEELTQDMELVETRPAGDGTFQKWASVVVPLGKEQNYTCRVYHEGLPHPLTLRWEGGIGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHDSMAEPKTVYWDRDMEQKLISEEDL

SEQ ID NO: 31 - DNA, 832 bp, Artificial Sequence
  Description of Artificial Sequence Synthetic nucleotide
gaattctacttcatacattttcaattaagatgcagttacttcgctgtttttcaatattttctgttattgcttcagttttagcagctagcttggataaaagagaacaatacaaattctactcagttcttaagggtggaccaggtggaggttcaggaggtggaggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctactacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagaggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagct

SEQ ID NO: 32 - protein, 542 aa, Artificial Sequence
  Description of Artificial Sequence Synthetic peptide
ILLHTFSIKMQLLRCFSIFSVIASVLAASLDKREQYKFYSVLKGGPGGGSGGGGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVERLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEGGIGSGGGGSGGGGSIQKTPQIQVYSRHPPENGKPNILNCYVTQFHPPHIEIQMLKNGKKIPKVEMSDMSFSKDWSFNILAHTEFTPTETDTYACRVKHDSMAEPKTVYWDRDMEQRLISEEDLHMQELTTICEQIPSPTLESTPYSLSTTTILANGKAMQGVFEYYKSVTFVSNCGSHPSTTSKGSPINTQYVFKDNSSTIEGRYPYDVPDYA

SEQ ID NO: 33 - DNA, 720 bp, Artificial Sequence
  Description of Artificial Sequence Synthetic nucleotide
gaattctacttcatacattttcaattaagatgcagttacttcgctgtttttcaatattttctgttattgcttcagttttagcagctagcttggataaaagagaacaatacaaattctactcagttcttaagggtggaccaggtggaggttcaggaggtggaggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctactacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagcggctccgcagatacctgaagaacgggaacgcgacgctgctgcgcacagattccccaaagg

SEQ ID NO: 34 - protein, 542 aa, Artificial Sequence
  Description of Artificial Sequence Synthetic peptide
ILLHTFSIKMQLLRCFSIFSVIASVLAASLDKREQYKFYSVLKGGPGGGSGGGGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVERLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEGGIGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHDSMAEPKTVYWDRDMEQKLISEEDLHMQELTTICEQIPSPTLESTPYSLSTTTILANGKAMQGVFEYYKSVTFVSNCGSHPSTTSKGSPINTQYVFKDNSSTIEGRYPYDVPDYA

SEQ ID NO: 35 - DNA, 1091 bp, Artificial Sequence
  Description of Artificial Sequence Synthetic nucleotide
gaattctacttcatacattttcaattaagatgcagttacttcgctgtttttcaatattttctgttattgcttcagttttagcagctagcttggataaaagagaacaatacaaattctactcagttcttaagggtggaccaggtggaggttcaggaggtggaggcccacactcgctgaggtatttcgtcaccgccgtgtcccggcccggcctcggggagccccggtacatggaagtcggctacgtggacgacacggagttcgtgcgcttcaacagcgacgcggagaatccgagatatgagccgcgggcgcggtggatggagcaggaggggcccgagtattgggagcgggagacacagaaagccaagggcaatgagcagagtttccgagtggacctgaggaccctgctcggctactacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagaggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagggtggaataggttcaggtggcggtggatcaggaggcggaggttcaatccagaaaacccctcaaattcaagtatactcacgccacccaccggagaatgggaagccg

SEQ ID NO: 36 - protein, 542 aa, Artificial Sequence
  Description of Artificial Sequence Synthetic peptide
ILLHTFSIKMQLLRCFSIFSVIASVLAASLDKREQYKFYSVLKGGPGGGSGGGGPHSLRYFVTAVSRPGLGEPRYMEVGYVDDTEFVRFNSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVERLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEGGIGSGGGGSGGGGSIQKTPQIQVYSRHPPENGKPNILNCYVTQFHPPHIEIQMLKNGKKIPKVEMSDMSFSKDWSFNILAHTEFTPTETDTYACRVKHDSMAEPKTVYWDRDMEQRLISEEDLHMQELTTICEQIPSPTLESTPYSLSTTTILANGKAMQGVFEYYKSVTFVSNCGSHPSTTSKGSPINTQYVFKDNSSTIEGRYPYDVPDYA

SEQ ID NO: 37 - protein, 9 aa, Artificial Sequence
  Description of Artificial Sequence Synthetic peptide
HYSPFRQLA

(19) United States
(12) Patent Application Publication
(10) Pub. No.: US 2003/0036506 A1
(43) Pub. Date: Feb. 20, 2003

(54) Mutated class I major histocompatibility proteins and complexes

(76) Inventors: David M. Kranz, Champaign, IL (US); Susan Brophy, Urbana, IL (US)

Correspondence Address: GREENLEE WINNER AND SULLIVAN P C, 5370 MANHATTAN CIRCLE, SUITE 201, BOULDER, CO, 80303, US

(21) Appl. No.: 10/015,535

(22) Filed: Dec. 10, 2001

Related U.S. Application Data
(60) Provisional application No. 60/254,495, filed on Dec. 8, 2000.

(51) Int. Cl. A61K 38/17; C07K 14/74; G01N 33/53; C12N 1/18

(52) U.S. Cl. 514/12; 435/7.1; 435/254.2; 530/350

(57) ABSTRACT

Provided herein are combinatorial libraries containing chimeric Major Histocompatibility Comples (MHC) Class I proteins displayed on the surfaces of recombinant yeast cells. Members of the libraries, especially where those libraries have been mutagenized either with error-prone Polymerase Chain Reaction or with site-directed oligonucleotide mutagenesis, are improved in conformation stability or in binding to a target, e.g., a peptide or other ligand as compared with the stability or binding affinity of a wild type MHC Class I chimeric protein. The improved mutant chimeric proteins can be selected by various means, including fluorescence activated cell sorting with a fluorescent ligand bound to the surfaces of the yeast cells displaying the improved mutant chimeric protein.

### CROSS REFERENCE TO RELATED APPLICATIONS

This application is a Continuation-in-Part of U.S. application No. 60/254,495, filed Dec. 8, 2000.

### ACKNOWLEDGMENT OF FEDERAL RESEARCH SUPPORT

This invention was made, at least in part, with funding from the National Institutes of Health (Grant No. PHS 5 RO1 AI35990). Accordingly, the United States Government has certain rights in this invention.

### BACKGROUND OF THE INVENTION

The field of the present invention is molecular biology, in particular, as it is related to combinatorial libraries of immune cell proteins displayed on the cell surface of a recombinant host cell. More specifically, the present invention relates to a library of major histocompatibility locus proteins displayed on the surfaces of recombinant yeast cells, to mutant MHC Class I and proteins selected for improved binding to particular target peptides, to mutant MHC Class I proteins selected for binding to a particular antigen, to MHC Class I proteins of improved stability and to the use of the selected high affinity and/or more stable MHC derivatives in diagnostic methods and imaging assays, among other applications including prophylactic and therapeutic treatments.

Proteins encoded by the major histocompatibility complex (called MHC proteins) are requisite components of the antigenic complexes that are involved in many diseases. These diseases include cases where the body reacts with one's own MHC proteins (in various autoimmune diseases) or infectious diseases and cancer, where the MHC are critical in binding and presenting foreign, antigenic peptides. In this invention, MHC proteins of the class I type were expressed as heterologous, surface-linked fusions on yeast cells with the goal of generating improved MHC proteins. Libraries of mutant MHC and mutant peptide-MHC complexes could be screened for higher surface levels in order to identify variants that exhibited improved properties, including enhanced stability. For the first time, this system allows the directed evolution of MHC molecules that represent novel agents for various diagnostic and therapeutic applications. These agents could be used in cancer, infectious diseases (e.g., virus infections), and autoimmune diseases (e.g., multiple sclerosis, type I diabetes, rheumatoid arthritis).

T cell receptors (TCRs) and antibodies have evolved to recognize different classes of ligands. Antibodies function as membrane-bound and soluble proteins that bind to soluble antigens, whereas in nature, TCRs function only as membrane-bound molecules that bind to cell-associated peptide/MHC antigens. All of the energy of the antibody:antigen interaction focuses on the foreign antigen, whereas a substantial fraction of the energy of the TCR:peptide/MHC interaction seems to be directed at the self-MHC molecule [Manning et al. (1998) Immunity 8:413:425]. In addition, antibodies can have ligand-binding affinities that are orders of magnitude higher than those of TCRs, largely because of the processes of somatic mutation and affinity maturation. In their normal cellular context, TCRs do not undergo somatic mutation, and the processes of thymic selection seem to operate by maintaining a narrow window of affinities [Alam et al. (1996) Nature 381:616-620]. The association of TCRs at the cell surface with the accessory molecules CD4 or CD8 also may influence the functional affinity of TCRs [Garcia et al. (1996) Nature 384:577-581]. Despite these differences, the three-dimensional structures of the two proteins are remarkably similar, with the hypervariable regions forming loops on a single face of the molecule that contacts the antigen.

There is a long felt need in the art for Class I MHC proteins and Class I MHC/peptide complexes with improved stability and/or with improved T cell regulatory properties. Such improved Class I MHC proteins or complexes are useful in activating T cells that participate in the removal of target cells including neoplastic cells and cells infected with pathogenic agents including, but not limited to, viruses, protozoans, bacteria, fungi or nematodes. The improved Class I MHC proteins and complexes of the present invention are also improved for use as research tools.

### SUMMARY OF THE INVENTION

The present invention provides combinatorial libraries of Class I MHC proteins displayed on the surfaces of recombinant host cells, for example, yeast cells, desirably, Saccharomyces cerevisiae. From such a library can be isolated mutant MHC proteins that exhibit a relatively high affinity for a peptide ligand of interest. Also within the scope of the present invention are methods for isolating mutant Class I MHC proteins with improved stability, especially as MHC/peptide complexes of improved stability.

The present invention further provides Class I MHC/peptide complexes and proteins that exhibit increased stability over the wild type Class I MHC/peptide complex or MHC protein, and which MHC proteins exhibit high affinity for a peptide ligand interest. This ligand can be a peptide, a protein, a carbohydrate moiety, or a lipid moiety, among others.

Suitable labels allowing for detection of a ligand bound to an MHC protein, directly or indirectly, include but are not limited to fluorescent compounds, chemiluminescent compounds, radioisotopes, chromophores, and others. The stable, MHC protein of the present invention, where it specifically binds to a tumor cell antigen with high affinity and specificity can be used in diagnostic tests to detect T cells that are specific for the Class I MHC/peptide complex. The Class I MHC/peptide complexes of the present invention can also be used to activate T cells and thus, to enhance an immune response to an antigen or target cell of interest.

Also provided by the invention are novel yeast display vectors and surface expression constructs in which the portion of the fusion protein that mediates attachment to the cell surface (the AGA2 sequence) is located downstream, or at the carboxy end of the protein/sequence of interest. See FIG. 2.

### BRIEF DESCRIPTION OF THE DRAWINGS

FIG. 1 provides a ribbon diagram corresponding to the crystal structure of a class I major histocompatibility complex with highlighted subunits. The MHC is trimeric. The MHC K^{b} α chain (^{˜}350 amino acids) binds to an 8-10 amino acid residue peptide in the peptide binding cleft. The β2 microglobulin (99 amino acids) associates non-covalently with the α chain.

FIG. 2 illustrates the displayed MHC protein of interest displayed on the yeast cell surface via a disulfide linkage through the AGA2 portion of the fusion protein comprising the MHC component.

FIG. 3 illustrates diagrammatically the pCT302 yeast surface display vector which contains a sequence encoding AGA2/HA-Class I MHC-c-myc fusion protein. This fusion protein coding sequence is expressed in yeast under the regulatory control of the GAL1-10 promoter. A similar vector, pYD1, is commercially available from Invitrogen.

FIG. 4 provides diagrams of various single-chain Class I MHC constructs cloned into the pCT302 Yeast Display Vector.

FIG. 5 provides the results of flow cytometric analyses of various K^{b} constructions. Yeast cells displaying K^{b}/β2m, SIYR K^{b}/β2m, dEV8 K^{b}/β2m, and OVA K^{b}/β2m were stained with anti-c-myc Mab 9E10 and biotinylated anti-K^{b} antibody (B.8.24.3) followed by FITC labeled F(ab′)₂ goat anti-mouse IgG or SA-PE (shaded peaks). For a background control, yeast were treated with only the secondary stain (unshaded peak). Labeled yeast were analyzed on Coulter Epics XL flow cytometer. All four constructions displayed the properly folded K^{b} molecule on the surface.

FIGS. 6A-6D illustrate histograms from CD69 up-regulation assays. Induction of T cell activation marker CD69 on naive splenocytes (10⁶) in the presence of K^{b}/β2m, anti-TCR scFv, and SIYR/K^{b}/β2m yeast cells. Induced yeast cells (10⁶) were incubated at 37° C. and 5% CO₂ with splenocytes from 2C TCR/RAG-1^{-/-} mice. After 20 hours in culture, cells were harvested, washed in PBS/0.5% BSA and stained with biotinylated clonotypic antibody, 1B2 [Kranz et al. (1984) Proc. Natl. Acad. Sci. USA 81: 7922-7926] for 45 minutes. These yeast were washed and stained with a mixture of the early activation marker FITC anti-CD69 antibody and SA:PE. The yeast/T cell mixture was analyzed for bound FITC-labeled anti-CD69 antibody by flow cytometry, gating on 1B2 positive T cells. The mean fluorescence units for FITC anti-CD69 antibody in the absence of yeast, presence of K^{b}/b2m yeast, anti-TCR scFv yeast, and SIYR/K^{b/}β2m yeast are indicated and illustrated in the histograms. Yeast cells that expressed K^{b} with the agonist peptide (called “SIYR”; SIYRYYGL SEQ ID NO:1), but not yeast cells with K^{b} and no peptide, were capable up-regulating the CD69 molecule on the 2C T cells. This up-regulation was even greater than observed with the positive control anti-TCR antibody KJ16 [Cho et al. (1998) J. Immunol. Methods 220(1-2): 179-188].

FIG. 7 shows the results of direct activation of T cells by yeast that express Class I Peptide/MHC, as measured by flow cytometry. Up-regulation on 2C TCR/RAG^{-/-} splenocytes incubated with varying ratios of yeast (3 to 100×10⁵ yeast and 106 T cells) were analyzed by flow cytometry. The mean fluorescence units of FITC anti-CD69 antibody was detected on 1B2 gated T cells following 20 hours of incubation (37° C., 5% CO₂) of 2C TCR/RAG^{-/-} splenocytes with yeast that expressed: SIYR/K^{b}/β2m, anti-TCR scFv (positive control), and K^{b}/β2m (negative control).

FIG. 8 shows direct activation of T cells by pMHC on yeast, thus providing evidence for pMHC specificity excess peptide and anti-K^{b} antibody inhibit CD69 up-regulation. In order to confirm the specificity of T cell activation, K^{b}/β2m (negative control), SIYR/K^{b}/β2m bearing yeast cells, and 2C TCR/RAG^{-/-} splenocytes were incubated with excess OVA (SIINFEKL, SEQ ID NO:2) peptide or anti-K^{b} antibody (50 μg/ml B.8.24.3) at 37° C., 5%CO₂. Excess OVA peptide binds to K^{b} and should compete for binding to the K^{b} molecule, but it is not recognized by 2C T cells. Anti-K^{b} antibody recognizes the al/a2 helices and should prevent binding by the T cell receptor from 2C T cells. After 1 hour, 2C TCR/RAG^{-/-} splenocytes were mixed with the yeast and incubated for 20 hrs. Detection of CD69 on 2C T cells following incubation with yeast in the absence or presence of inhibitors was detected with FITC anti-CD69 antibody and reported as the mean fluorescence units. Both excess OVA peptide and the anti-K^{b} antibody inhibited the up-regulation of CD69, confirming that the recognition of the yeast-bound SIYR/K^{b} was specific.

FIG. 9 diagrammatically illustrates cloning and transformation via homologous recombination. Linear mutagenized MHC protein coding sequence and linearized pCT302 vector are co-electroporated into yeast. This strategy is used in the preparation of the yeast display library for the MHC Class I protein.

FIG. 10 illustrates the strategy for mutagenizing a yeast display library and screening that library by taking advantage of the binding properties of mutant vs wild type displayed proteins. Fluorescently labeled ligands or antibodies which bind the displayed protein are employed in flow cytometry assays.

FIG. 11 provides examples of sorting libraries generated by random mutagenesis. A SIYR/K^{b}/β2m error prone library (SEP) and a dEV8/K^{b}/β2m error prone library (dEP) were each sorted 3 times. The SEP and dEP yeast (10⁷), after having been induced for 2 days, were washed with PBS/0.5% BSA, stained with biotinylated anti-K^{b} (B.8.24.3) for 60 min, washed again, and stained with SA:PE for 30 min. The first sort isolated the top 1.0% of the population using a 1:25 dilution of anti-K^{b}. The second and third sorts were stained with a 1:250 and 1:500 dilution of anti-K^{b} for SEP and 1:250 dilution for both the second and third sort of dEP. The second and third sorts isolated the top 0.25% and 0. 1% of the population, respectively. Representative histograms from the second and third sort of the SEP library illustrate the mean fluorescence shift of the total population or enhancement of the more fluorescent yeast cells from sort 2 to sort 3. This indicated that there were likely to be K^{b} mutants that exhibited increased stability and hence, increased surface levels [Shusta, (1999) J. Mol. Biol. 292: 949-956].

FIG. 12 shows the K^{b} surface levels of mutant clones isolated by sorting from dEV8/K^{b} error prone PCR library. Following sorting, ten randomly selected clones from the dEV8/K^{b/}β2m library were analyzed on the flow cytometer for binding to the biotinylated anti-K^{b} antibody (B.8.24.3). Binding was detected with SA:PE, and mean fluorescence for each clone was determined.

FIG. 13 shows sequences of mutant clones isolated by sorting from dEV8/K^{b} error prone PCR library. The mutations in three clones selected from the dEV8/K^{b}/β2m error prone library (sort 3) and the crystal structure with two of the mutations (W167R,Y63N) are shown.

FIG. 14 illustrates construction of a directed, mutagenic peptide library in dEV8/K^{b} library construction. Mutagenic PCR of positions 1-3 of the peptide dEV8 was performed using the degenerate upstream primer (5′ ATA TTT TCT GTT ATT GCT TCA GTT TTA GCA GCT AGC TTG GAT AAA AGA NNS NNS NNS AAA TTC 3′, SEQ ID NO:3) and a downstream vector-specific primer. Using homologous recombination, the mutagenic dEV8/K^{b}/β2m PCR product and Nhe I-Nde I digested dEV8/K^{b}/β2m pCT302 were electroporated into electrocompetent yeast (BY5465, strain EBY100). A dilution of the transformed library was plated on SD-CAA plates and incubated for 3 days at 30° C. to obtain the library size (7×10³).

FIG. 15 shows the properly folded α3 domain, but the α1/α2 domains of L^{d} are not displayed on yeast. L^{d}/β2m yeast cells were pulsed for two hours with the L^{d}-binding peptide QL9 (QLSPFPFDL, SEQ ID NO:4), washed with PBS/0.5%BSA, and stained with anti-L^{d} antibodies, 28.14.8 (α3 specific) or 30-5-7 (α1/α2 specific). After 45 min, cells were washed and stained with FITC-labeled F(ab=)₂ goat anti-mouse IgG, and binding was detected using flow cytometry. The histograms generated from the L^{d}/β2m displaying yeast represent the binding of the α3 specific antibody (28.14.8). The 30-5-7 antibody, α1/α2 specific, did not show the same binding (histograms not included), suggesting that at least one of these two domains was not folded properly and thus might be stabilized by a process of directed evolution and yeast display.

FIG. 16 illustrates the result of sorting a yeast display mutant library of “unstable” Class I MHC L^{d}. Yeast cells displaying L^{d}/β2m were induced at 20 C for 2 days in the presence of an L^{d}/β2m specific peptide, QLSPFPFDL (QL9, SEQ ID NO:4). L^{d}/β2m yeast were stained with supernatants from D. melanogaster cells expressing the high-affinity T cell receptor (m6), biotinylated anti-T cell receptor antibody (F23.1) and a streptavidin-phycoerythrin (SA:PE) conjugate (shaded peak). The L^{d}/β2m yeast cells were also stained with an α2 domain specific antibody (30-5-7) and an α3 domain specific antibody (28.14.8) followed by a FITC labeled F(ab′)₂ goat anti-mouse IgG (shaded peak). For a background control, yeast cells were treated with only the secondary stains (unshaded peaks). Labeled yeast cells were detected using a Coulter EPICS XL flow cytometer. The L^{d}/β2m α3 domain was folded properly on the surface, but not the α2 domain or the TCR binding domain.

FIG. 17 demonstrates that the L^{d} mutant 30.8 exhibits a properly folded TCR binding domain and properly folded α2/α3 domains. An L^{d}/β2m error prone library was created using homologous recombination and electroporation into electrocompetent S. cerevisiae EBY100 cells. The L^{d}/β2m error prone yeast library was sorted with L^{d}/βb 2m α3 specific antibody, 28.14.8, and an L^{d}/β2m α2 specific antibody, 30-5-7. Sorted yeast cells were screened by flow cytometry for binding to 30-5-7 antibody and high affinity T cell receptor supernatant (m6), as described in FIG. 16 above. Unlike yeast cells that express the wild type L^{d}/β2m, the L^{d}β2m mutant, yeast cells expressing 30.8 were detected by the α2 specific antibody (30-5-7) and by the high affinity T cell receptor (m6) when the correct peptide (QL9) was added exogenously. Mutant 30.8 was also detected by the α3 specific antibody, 28.14.8.

FIG. 18 summarizes the results for a yeast display library of peptides that bind to MHC class I K^{b}. Known K^{b} peptides (SIYRYYGL, SEQ ID NO:1; EQYKFYSV, SEQ ID NO:5; SIINFEKL, SEQ ID NO:2) have anchor residues at peptide positions 5 and 8. P5 requires an aromatic amino acid whereas P8 requires a hydrophobic amino acid. Yeast displaying a K^{b} peptide binding motif, but with the AGA2 fused at the amino terminus, were isolated after incubation of yeast cells with the dEV8 (EQYKFYSV)/K^{b} tetramer.

FIG. 19 shows that yeast cells displaying the K^{b} peptide motif were detected by flow cytometry using a fluorescent-labeled K^{b} tetramer. Yeast cells with the C-terminal sequence shown in the top panel were stained with dEV8 (EQYKFYSV)/K^{b} tetramers for 12 hours. The dEV8 peptide dissociates from the K^{b} pocket, and the AGA2-fused peptide on the yeast surface binds to the free K^{b}. Binding of the peptide motif to the K^{b} tetramer was detected by flow cytometry (shaded peak). For a background control, yeast cells were treated with streptavidin-phycoerythrin (SA:PE) conjugate alone. Mutagenic substitution of the proline residue at position P4 with an alanine residue reduced binding significantly (bottom panel), indicating that the prolione is important in K^{b}-binding.

FIG. 20 shows that novel class I MHC binding peptides are isolated by yeast display technology. Using the lead peptides identified by yeast display experiments (FIG. 19), one can now identify improved peptides by production of libraries that contain mutations in non-anchor residues (e.g. P6, P7, and P9).

### DETAILED DESCRIPTION OF THE INVENTION

The role of proteins encoded by the major histocompatibility complex (called MHC proteins) has now been known for almost twenty years. MHC proteins are expressed by every individual and function as “antigen-presenting” molecules. That is, each MHC protein can bind to a variety of different small peptides (8 to 20 amino acids in length) that are derived from proteins present inside a cell. MHC proteins present both self-peptides (i.e., derived from an individual's own endogenous proteins) and foreign peptides (i.e., derived from a foreign agent such as a virus). Once a peptide is bound to the MHC protein, the entire peptide-MHC complex is expressed on the surface of the cell. If the peptide is foreign, a T lymphocyte (T cell) can potentially recognize the complex, and the resultant interaction of the T cell receptor (TCR) and the pMHC can result in T cell activation. T cell activation can lead to recruitment of other immune cells and a corresponding inflammatory reaction. Such inflammatory reactions are beneficial if the pMHC target antigen is, in fact, derived from an infectious agent or from a transformed cell (i.e., cancer). However, such inflammatory reactions can be very detrimental if the pMHC target antigen is derived from self tissue, as the reactions can lead to severe autoimmune disease, where an individual's immune system attacks normal tissue. Such is the case when a patient's lymphocytes attack the islet cells of the pancreas (type I diabetes), the nervous system (multiple sclerosis), or joint-derived components (rheumatoid arthritis).

The central role of pMHC complexes in these phenomena has been established by thousands of published studies that include genetic linkages of diseases to the human MHC locus (HLA). It has now also been established that it is possible to use appropriately characterized peptide-MHC molecules as either agonists of an immune response (e.g., in cancer and infectious diseases) or as antagonists of responses (e.g., in autoimmune responses). While several approaches have been taken to produce such pMHC complexes in soluble forms for these purposes and for biochemical/structural studies, it has not been possible to use current methods of in vitro directed evolution to improve the stability or antigenicity of the pMHC complex. This is because the pMHC complex is normally a membrane-associated complex composed of multiple different subunits (heavy chain, beta-2-microglobulin, and peptide in the case of a class I MHC and α-chain, β-chain, and peptide in the case of class II MHC) and such proteins are typically not amenable to the current methods of directed evolution (primarily phage display). The present invention shows that a display system for directed evolution can be used to express properly folded Class I MHC proteins on the surface of yeast. The displayed peptide-MHC complexes can be used to directly activate T cells, in order to identify/screen for pMHC antigens. In addition, mutated libraries of the pMHC proteins could be created and used for selection by flow sorting of stabilized pMHC variants. The stabilized variants can be identified because they were expressed at higher levels on the yeast surface and can therefore be easily identified by using a fluorescent-labeled probe for the pMHC construct, combined with high-throughput flow cytometric sorting or such cells.

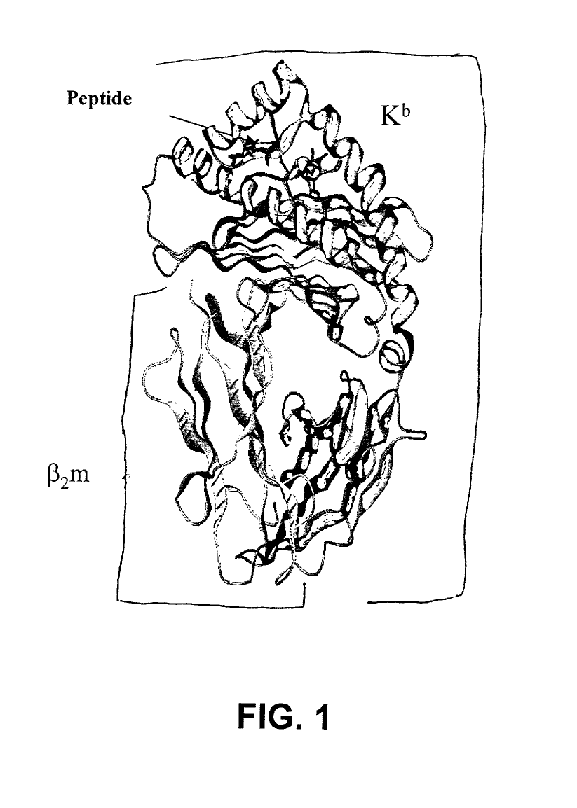

The Class I MHC proteins (see FIG. 1 for ribbon diagram) are composed of an α chain of 350 amino acids and a β2m chain of 99 amino acids; the two chains are noncovalently associated with one another. Peptides that are about 8-10 residues bind the Class I MHC molecules. About 10⁵ to 10⁶ peptide-Class I MHC complexes are displayed on the surfaces of nucleated cells. Class I MHC complexes are involved in the recognition of virus infected cells, pathogen-infected cells, and tumor antigens by cytotoxic T lymphocytes.

To date, no Class I MHC protein has been crystallized without a peptide bound in the peptide binding cleft. Some Class I MHC proteins are difficult or impossible to produce in soluble form because of their instabilities. For example, the mouse Class I MHC K^{b} is relatively stable, and it has been used in many studies. By contrast, the mouse Class I MHC L^{d} is less stable, and it has been more difficult to produce. The stability of a particular peptide-Class I MHC complex is directly related to its ability to stimulate efficient T cell responses, for example, in vaccine applications. A system for the in vitro evolution of more stable peptide-MHC complexes allows for novel agents and vaccines to be used in stimulating protective immune responses against diseases and neoplastic conditions.

The present invention allows the creation and isolation of stabilized variants of peptide-MHC complexes. Toward this end, we have displayed single-chain peptide/Class I MHC (α chain/β2m) complexes on the surface of yeast cells. Yeast cells expressing a specific peptide/MHC complex (SIYR/K^{b}) on the cell surface were capable of directly activating T cells, thus providing a method for screening powerful T cell agonists. Mutant libraries of a less stable peptide/MHC complex (dEV8/K^{b}) were made and expressed, and mutant MHC polypeptides of increased stability were isolated. Similarly, the less stable MHC L^{d} was expressed as a mutant library displayed on the surface of recombinant yeast cells, and more stable L^{d} variants were isolated using flow cytometry screening methodology. WO 99/36569, incorporated by reference herein, provides abundant discussion of this display technology.

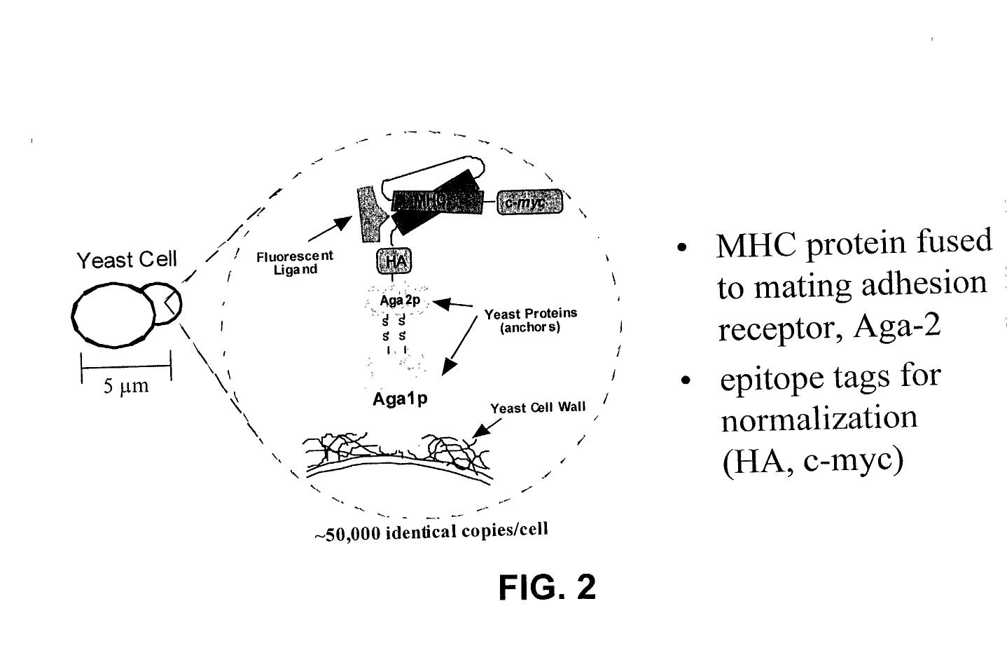

FIG. 2 illustrates the MHC protein of interest displayed on the yeast cell surface via a disulfide linkage through the AGA2 portion of the fusion protein comprising the MHC component. AGA2 is a mating adhesion receptor which is naturally bound to the cell surface in disulfide linkage to the AGAL protein. The HA and the c-myc portions of the displayed fusion protein serve as epitope tags and can be used in normalizing the fluorescent peptide binding data. Each recombinant yeast cell displays about 50,000 copies of the surface-bound fusion protein (if stable) on its surface. A fluorescent antibody or peptide ligand is added, and the cells are sorted using flow cytometry. Those MHC fusion proteins of increased stability exhibit stronger binding of the fluorescent ligand, and these cells are selected during the cell sorting procedure.

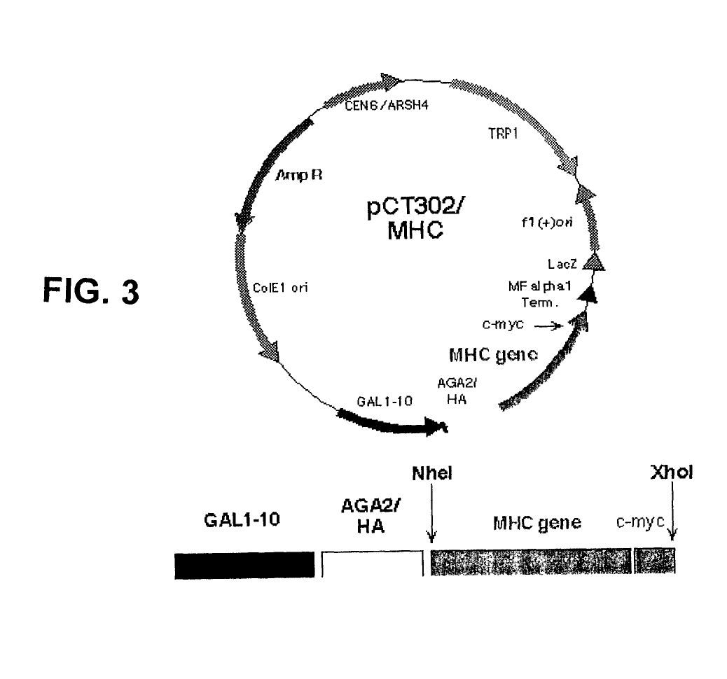

FIG. 3 illustrates diagrammatically the pCT302 yeast surface display vector which contains a sequence encoding AGA2/HA-Class I MHC-c-myc fusion protein. This fusion protein coding sequence is expressed in yeast under the regulatory control of the GAL1-10 promoter. A similar vector, pYD 1, is commercially available from Invitrogen (Carlsbad, Calif.).

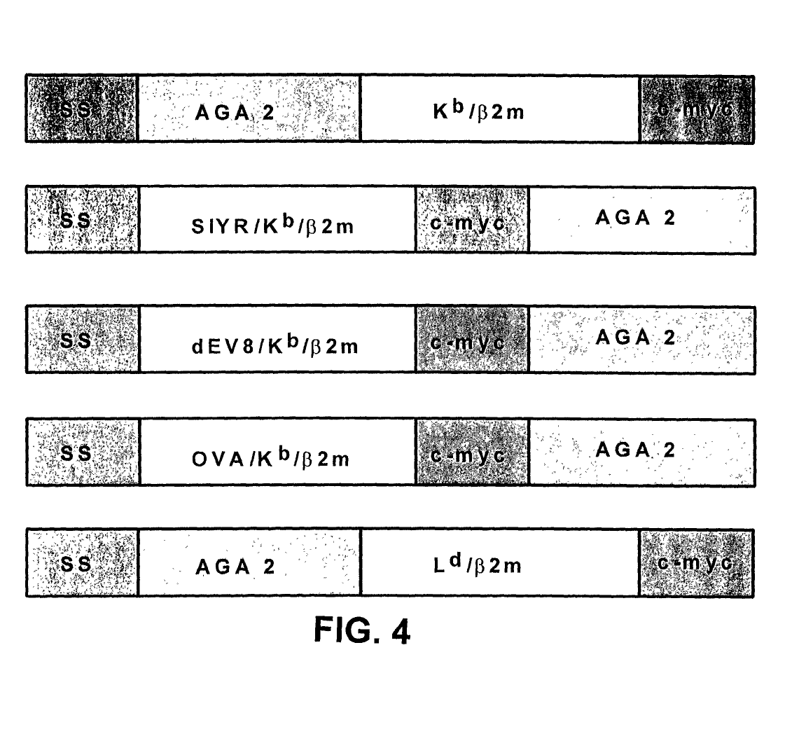

FIG. 4 shows various constructs for the expression of fusion proteins containing portions representing the peptide binding portions of various Class I MHC proteins. Surprisingly, the AGA2 portion was functional either at the N-terminus or the C-terminus of the fusion protein, and it mediated binding to the yeast cell wall surface when associated with any of the protein portions tested and in both the amino- or carboxyl terminal positions. In FIG. 4 SS refers to a signal peptide sequence necessary for proper intracellular transport of the fusion protein. The signal peptide is cleaved prior to display on the cell surface. The MHC polypeptide coding portion includes a sequence encoding a 15 amino acid spacer between the COOH terminus of the α chain and the NH₂ end of the P2m portion. HA refers to the peptide tag derived in sequence from hemagglutinin, which tag is located at the amino terminus of the a chain. C-myc refers to the peptide tag at the COOH terminus of the β2m portion. The three peptides linked at the amino terminus of K^{b} represent a strong agonist (“SIYR” peptide, SEQ ID NO:1), a weak agonist (dEV8) and a null peptide (OVA) for the T cell clone called 2C. The AGA2 coding sequence was cloned at the COOH termini of these constructs in order to allow free NH₂ termini of the peptides, which is generally thought to be important for proper binding to Class I MHC polypeptides. All fusion protein coding sequences were assembled using standard polymerase chain reaction (PCR) strategies.

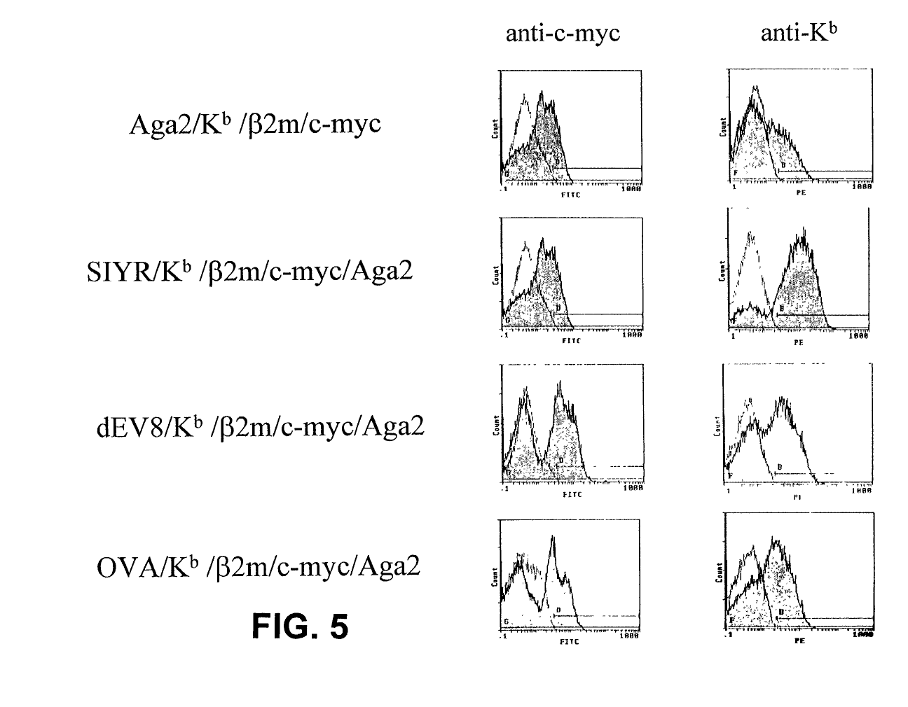

The correct foldings of various fusion display proteins were confirmed by fluorescence activated cell sorting after binding of the recombinant yeast cells to fluorescently labeled antibody specific for c-myc or K^{b}. See FIG. 5.

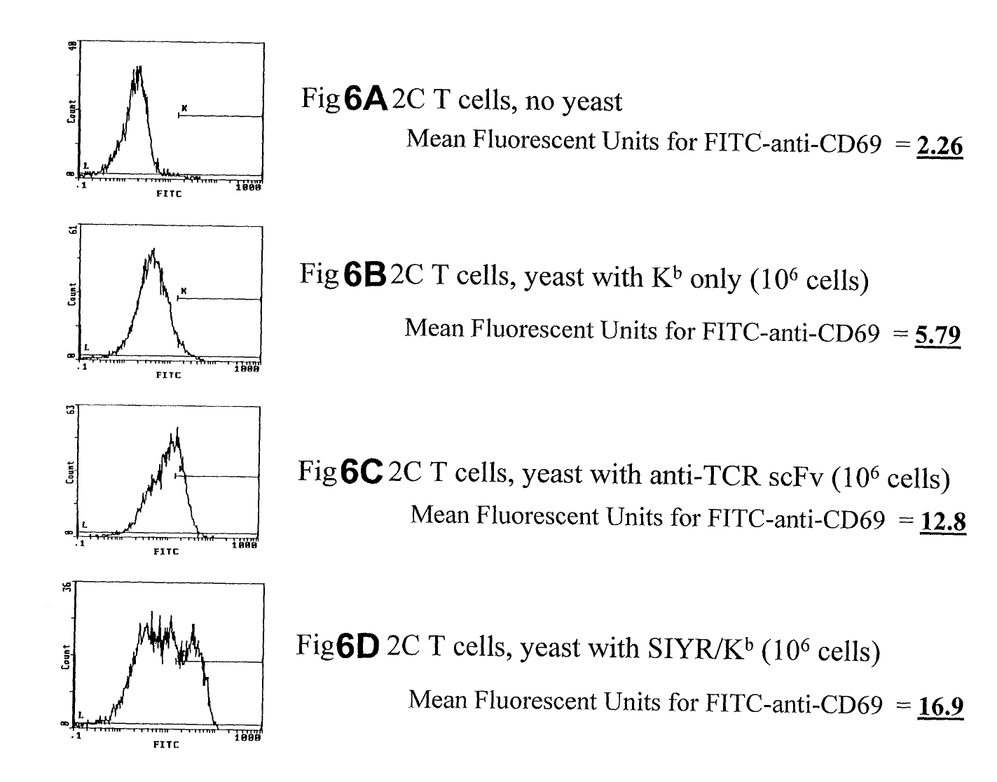

To demonstrate the direct activation of T cells by recombinant yeast cells expressing a peptide/MHC complex, naive splenic T cells from a TCR transgenic mouse (2C TCR tg) were incubated with yeast cells expressing SIYR/K^{b}, K^{b} (negative control) or anti-TCR scFv (positive control). After about 20 hours in culture, the T cells were analyzed by flow cytometry for the up-regulation of the activation marker CD69. See FIGS. 6A-6D for a graphical display of the results.

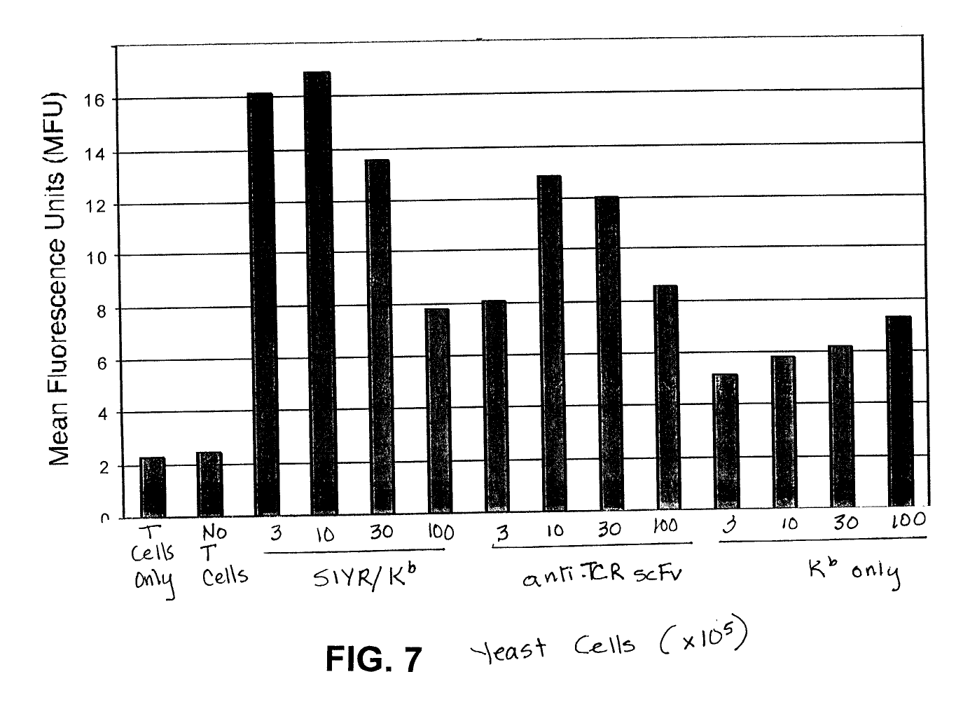

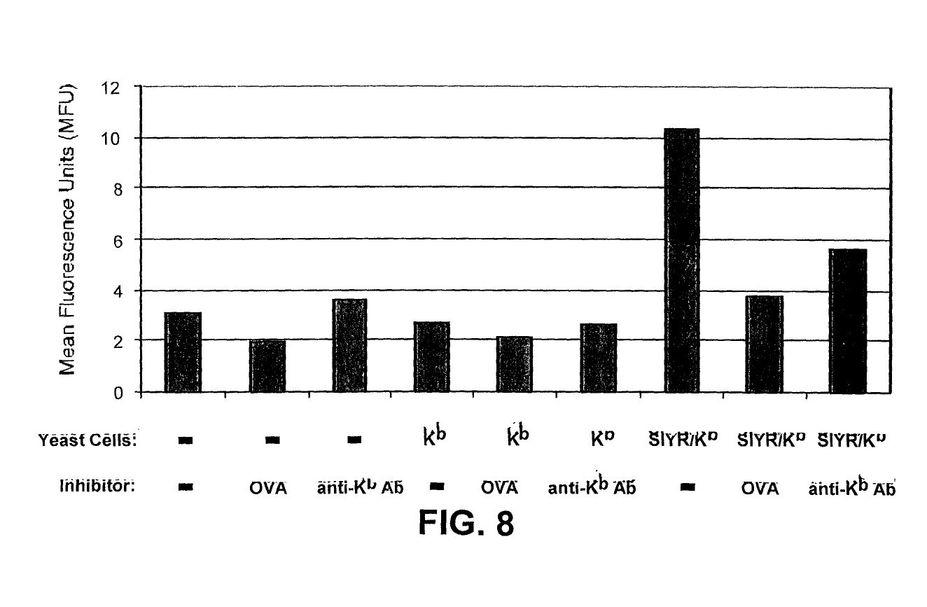

FIG. 7 shows the dependence of T cell activation on T cells and on the presence of a peptide/MHC complex. FIG. 8 demonstrates the pMHC specificity: excess free peptide and anti-K^{b} antibody inhibit CD69 up-regulation in T cells.

We have demonstrated direct activation of T cells by yeast cells that express peptide/MHC. Splenocytes (10⁶) from 2C TCR/RAG-1^{-/-} mice were combined with approximately 3-100×10⁵ of KF washed yeast cells displaying K^{b}/β2m, SIYR/K^{b}/β2m, or anti-TCR scFv (positive control) at 37 C, 5% CO₂ in a 24 well plate. After 20 hours in culture, cells were harvested, washed in PBS/0.5%BSA and stained with biotinylated clonotypic antibody, 1B2 [Kranz et al. (1984) supra] for 45 minutes. These yeast/T cell mixtures were washed and stained with a mixture of the early activation marker FITC-labeled anti-CD69 antibody and SA:PE. The yeast/T cell mixture was analyzed for the CD69 marker (present only on activated T cells) by flow cytometry and gating on 1B2 positive T cells.

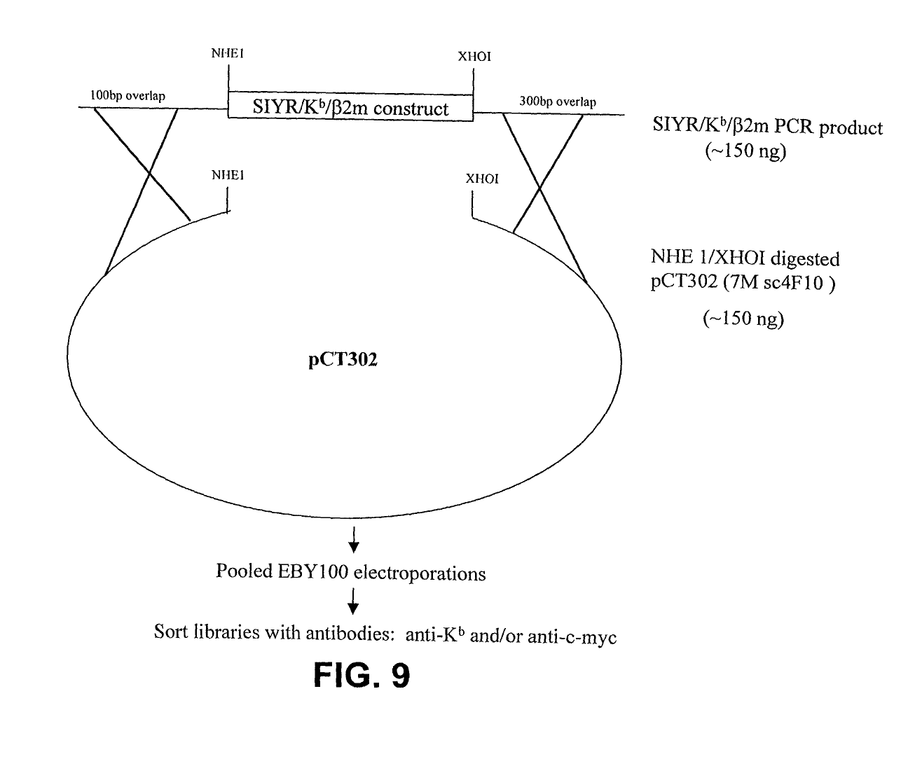

The yeast display system was exploited to produce a random mutagenized library from which stabilized mutant Class I MHC K^{b} sequences were isolated. Constructs encoding the fusion proteins containing SIYR/K^{b} and dEV8/K^{b} portions were mutagenized randomly using error prone PCR (0.16 Mn:Mg molar ratio). The homologous recombination scheme illustrated in FIG. 9 was employed to create the libraries. From the SIYR/K^{b} experiment, 3.6 million transformants were recovered. From the dEV8/K^{b} experiment, 2.7 million transformants were recovered. 5 plasmid inserts from each mutated library were sequenced (about 3200 bp of sequence per library) to determine the mutation frequency. In the SIYR/K^{b} experiment the mutation frequency was 0.37% (2 wild type sequences, 3, 4, and 5 mutant sequences). In the dEV8/K^{b} experiment, there was a 0.06% mutation frequency (4 wild type, 1 mutant sequence).

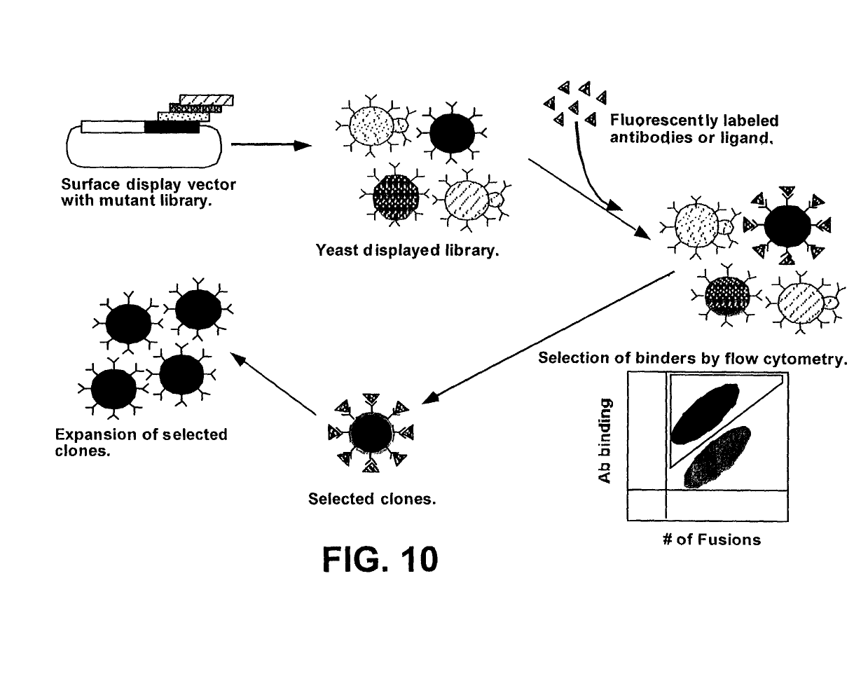

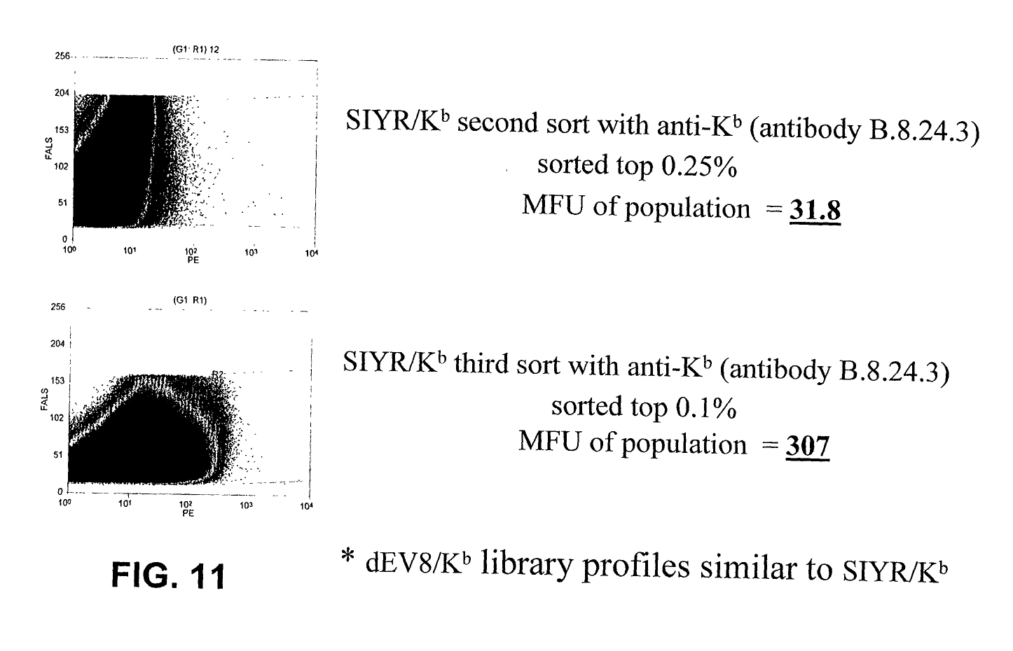

FIG. 10 diagrammatically presents the generic screening strategy for screening the mutated yeast display libraries. FIG. 11 illustrates the results for second and third sorts with anti-K^{b} monoclonal antibody B.8.24.3. In the second sort the top 0.25% of the cells (according to fluorescence intensity, mfu, mean fluorescence units) were selected, and in the third sort, the top 0.1% of the cells were selected. The profiles were similar for the dEV8/K^{b} experiment.

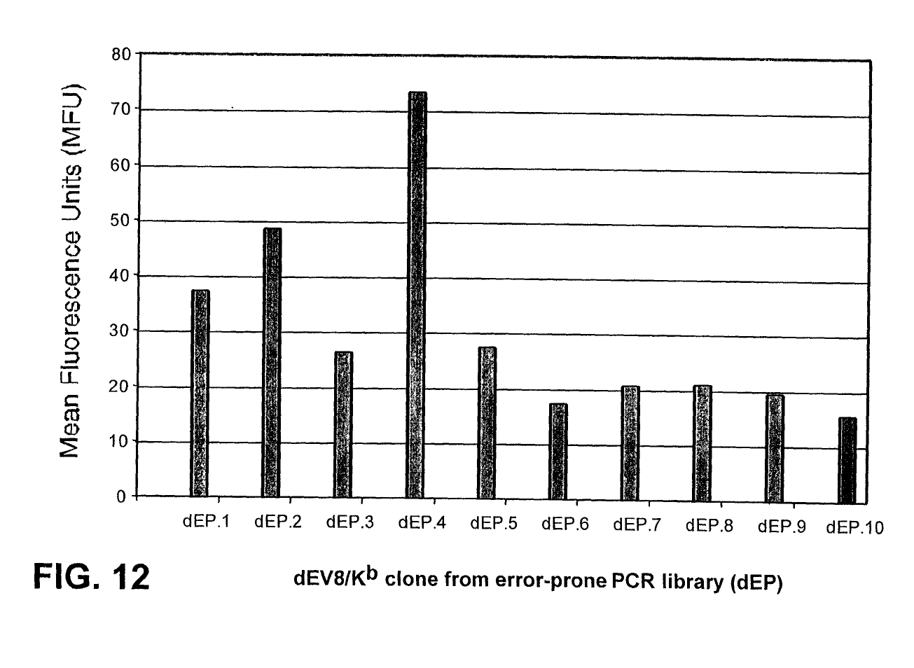

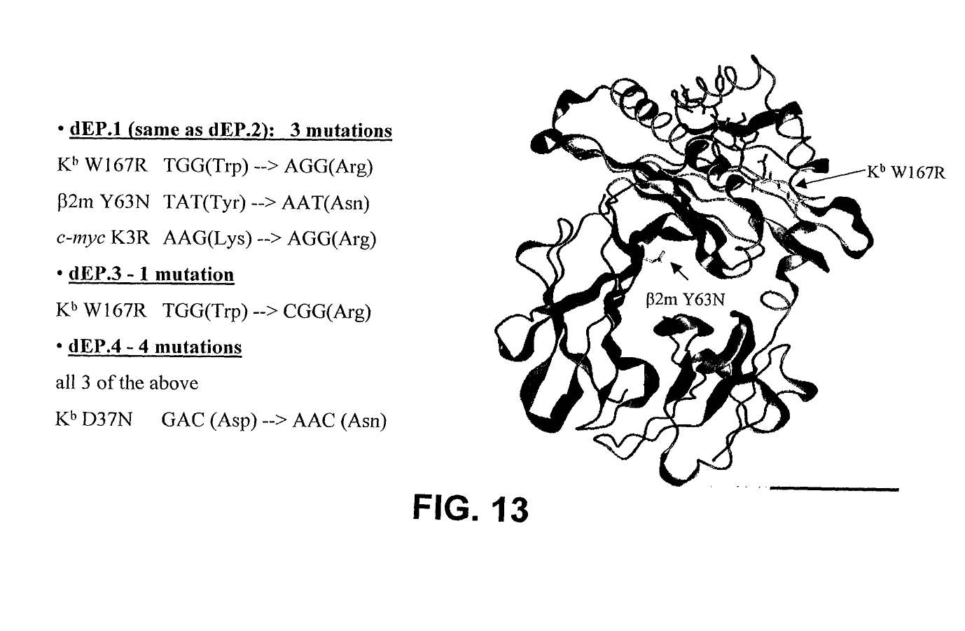

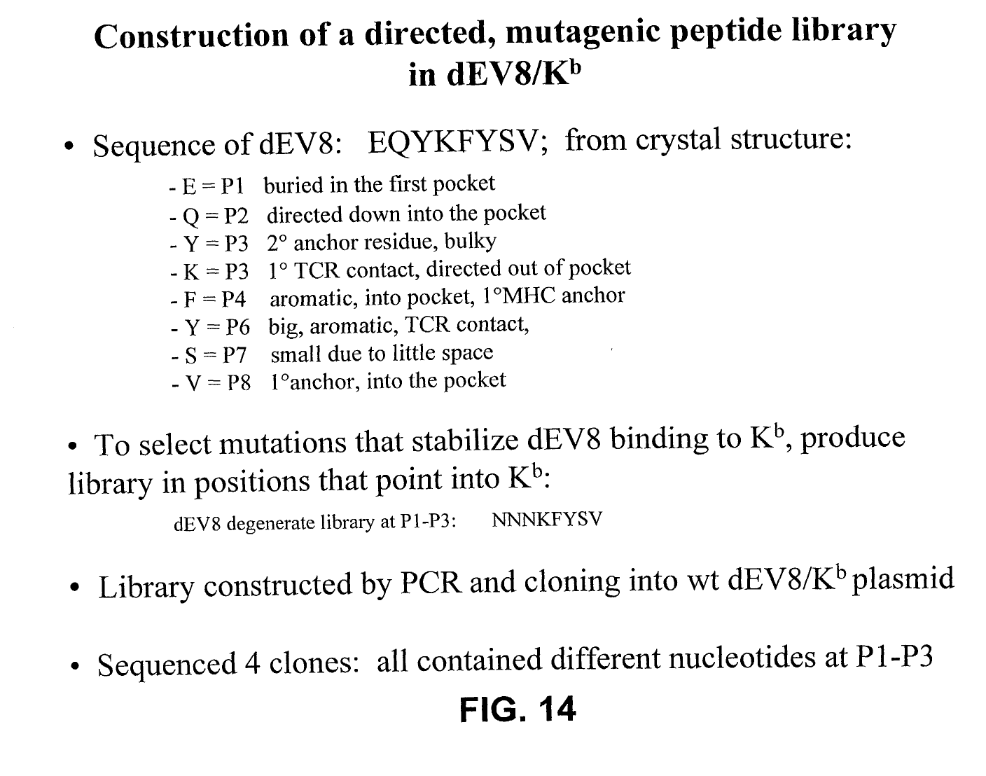

FIG. 12 illustrates different K^{b} binding as reflected in different levels of fluorescent peptide bound after sorting of the dEV8/K^{b} mutant library produced using error prone PCR. FIG. 13 presents selected sequences of mutant clones isolated by sorting of the dEV8/K^{b} mutant library, and the positions of the mutations are shown on the ribbon diagram of the Class I MHC protein. The following provides further details on the construction of a dEV8/K^{b} mutant library. From the ribbon diagram it is deduced that the sequence EQYKFYSV (SEQ ID NO:5) is important. The E (P1, the first amino acid of the peptide sequence) is buried in the first pocket. Q, P2, is directed down into the pocket. Y, P3, is a bulky, secondary anchor residue. K, P3, is a primary TCR contact, and it is directed out of the pocket. The P residue (P4) is an aromatic residue directed into the pocket; it serves as a primary MHC anchor. The Y residue at P6 is a large aromatic residue which also functions in TCR contact. The S residue (P7) is a small residue; the small size is dictated by the space available. The last V residue (P8) serves as a primary anchor residue, and it is directed into the pocket. To select mutations that stabilize dEV8 binding to K^{b}, mutations are introduced in the library at positions that point into K^{b}. A degenerate library is produced at P1-P3 (i.e., NNNKFYSV, SEQ ID NO:6). That library is constructed by error prone PCR and the mutant library is introduced into the wild type dEV8/K^{b} plasmid by homologous transformation as shown in FIG. 9. Isolated recombinants are then sequenced. Of four sequenced, each contained different nucleotides encoding P1-P3. See FIG. 14 and its description herein above.

The motivations for using directed evolution to isolate stabilized variants of L^{d} include the lower stability of the wild type L^{d} protein based on biochemical studies, the suboptimal loading of peptides in the endoplasmic reticulum, the slower intracellular transport of L^{d} (4 hr v 1 hr), the lower cell surface expression (2-4 fold lower) and fewer α/β2m contacts. See Table 1.

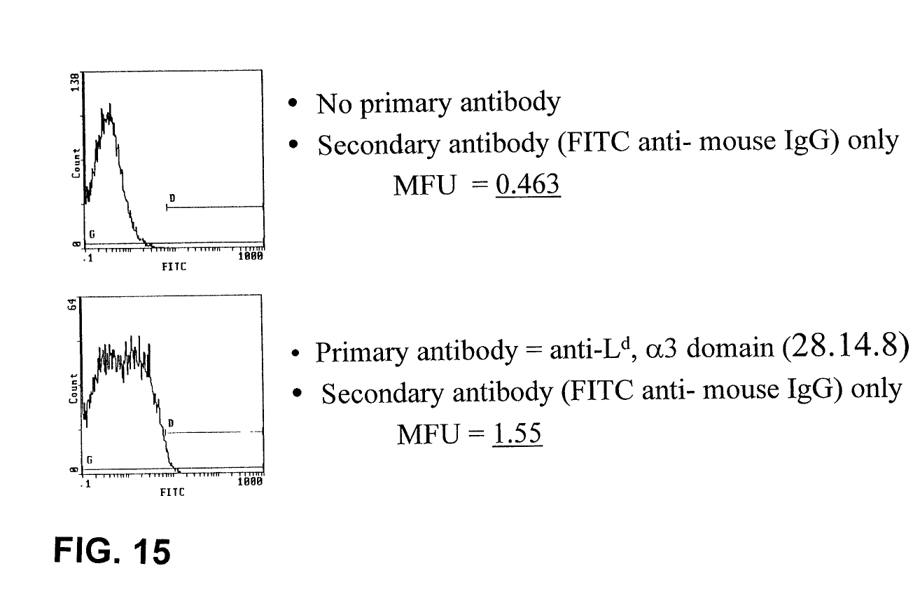

FIG. 15 provides data demonstrating that the properly folded α3 domain but not the α1/α2 domains of L^{d} are displayed on the surface of recombinant yeast cells. We have also demonstrated that anti-L^{d} α½ domain (monoclonal antibody 30.5.7) did not bind to the same L^{d}/β2m displayed on yeast cell surfaces.

In summary, the Class I MHC K^{b} was cloned as an AGA2 fusion in single chain format (AGA2-HA-K^{b}-β2m-c-myc). The full protein was detected on the yeast cell surface using an antibody that recognizes the properly folded α1/α2 domains of K^{b}. The coding sequences of this protein were also cloned with AGA2 at the carboxy terminus, with K^{b} binding peptides (SIYR, dEV8 and OVA) linked at the amino termini (peptide-HA-K^{b}-β2m-c-myc-AGA2). Yeast that expressed the T cell specific peptide K^{b} on the cell surface were capable of directly activating T cells, thereby providing a system for screening T cell agonists.

To create stabilized K^{b} mutant fusion protein, two different mutational libraries were produced by error prone PCR to yield random mutations after subsequent homologous recombination of mutagenized coding sequence and linearized vector after co-electroporation. Mutants that displayed higher yeast surface levels, and thus presumptive enhanced stability, were identified by flow cytometry sorting.

The unstable class MHC protein L^{d} was also cloned as an AGA2 fusion in single chain format (AGA2-HA-L^{d}-β2m-c-myc). L^{d} was detected on the yeast cell surface with an α3-specific antibody, but properly folded α1/α2 domains were not detected with anti-L^{d} antibody, suggesting that stabilized variants of L^{d} can be engineered by directed evolution and flow sorting for improved yeast surface expression.

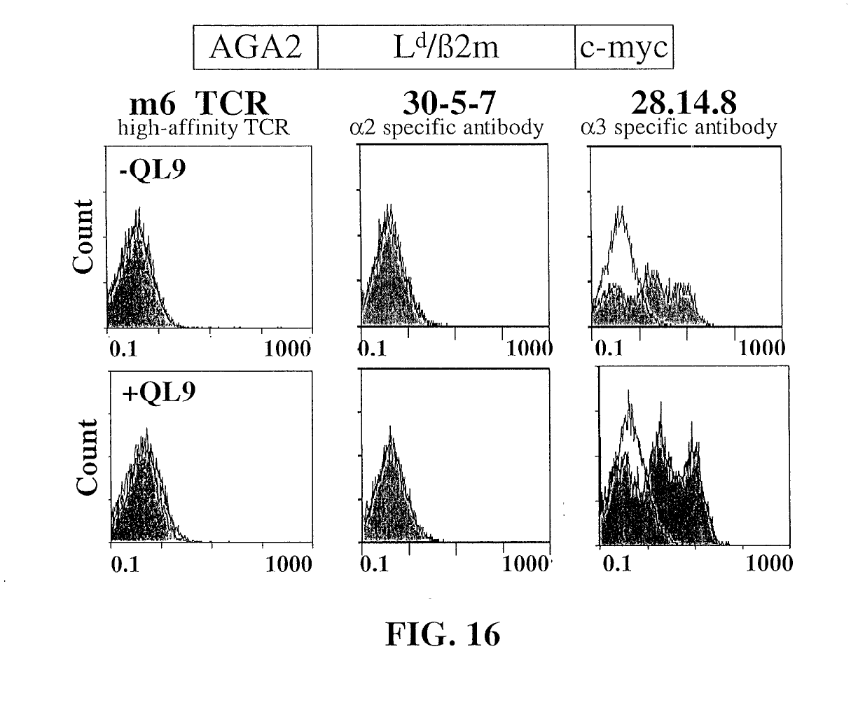

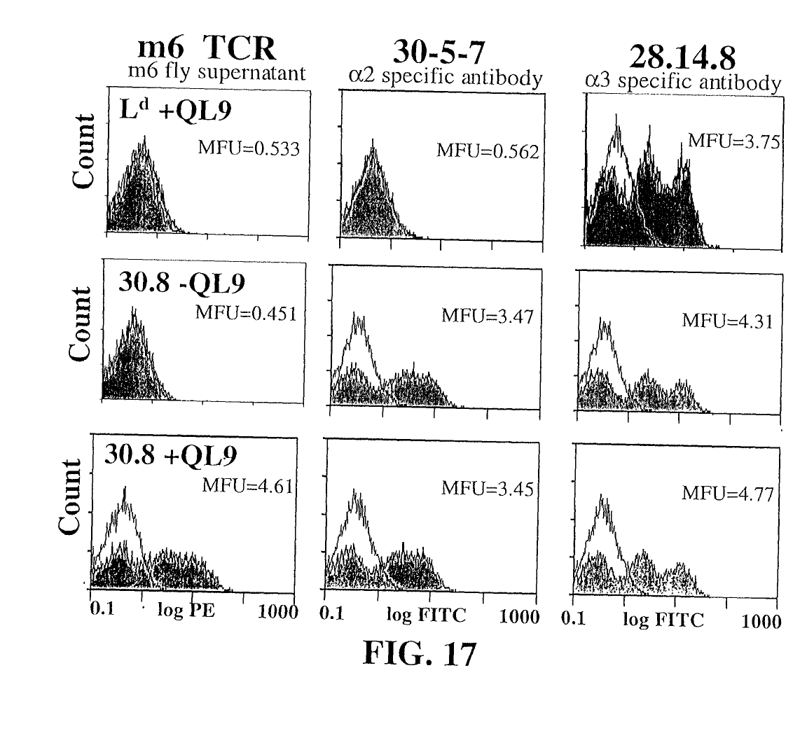

The present inventors have now succeeded in isolating stabilized mutants of the class I molecule L^{d}. The L^{d} molecule has been shown to be relatively unstable. When expressed in the yeast display system, only the α3 domain appears to be folded properly on the surface (FIG. 16). The displayed protein is not recognized by a conformation-specific α2 antibody (30-5-7) nor is the QL9/L^{d} complex recognized by the high-affinity T cell receptor called m6 (FIG. 16). To isolate stabilized L^{d}, a library of random mutants was expressed in yeast, and the library was selected with anti-L^{d} antibody 30-5-7; see FIG. 17 and its description. Various mutants were isolated. One mutant (30.8) is shown in FIG. 17. Mutant 30.8 bound to both the α2-specific antibody and the QL9/L^{d} (30.8) complex bound to the high affinity TCR m6, reflecting increased stability over its wild type parent protein.

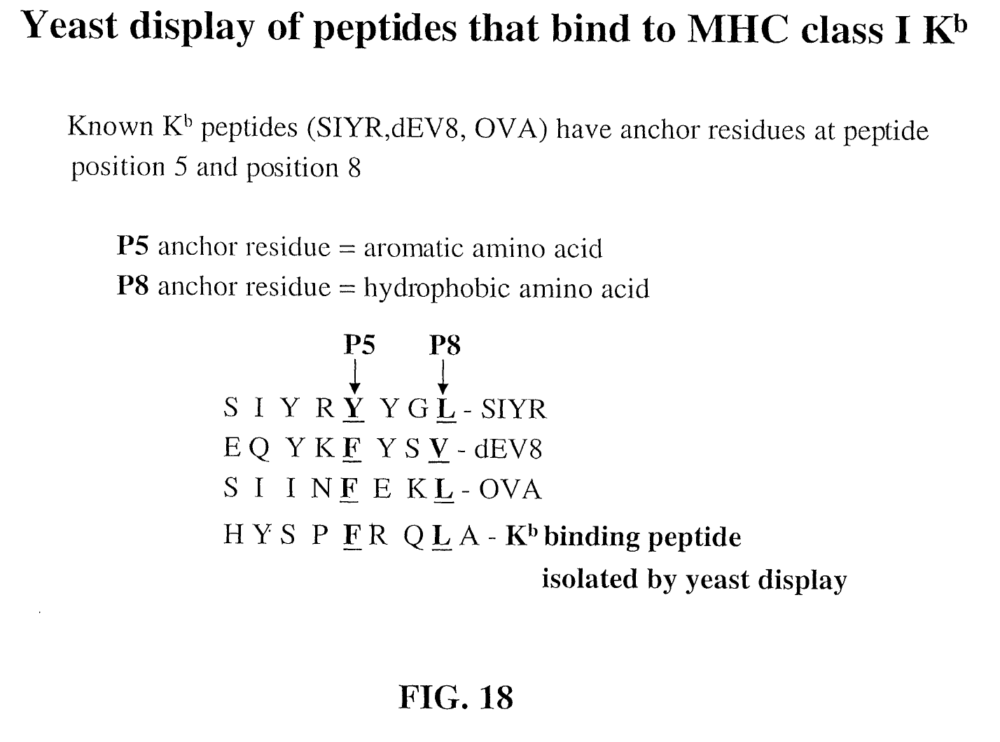

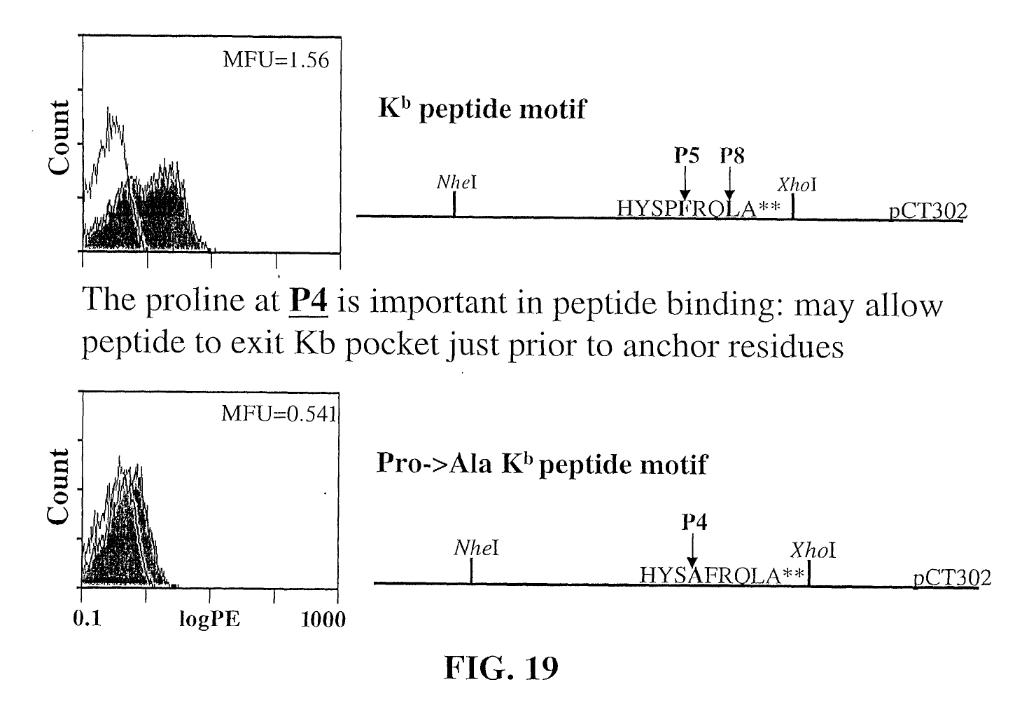

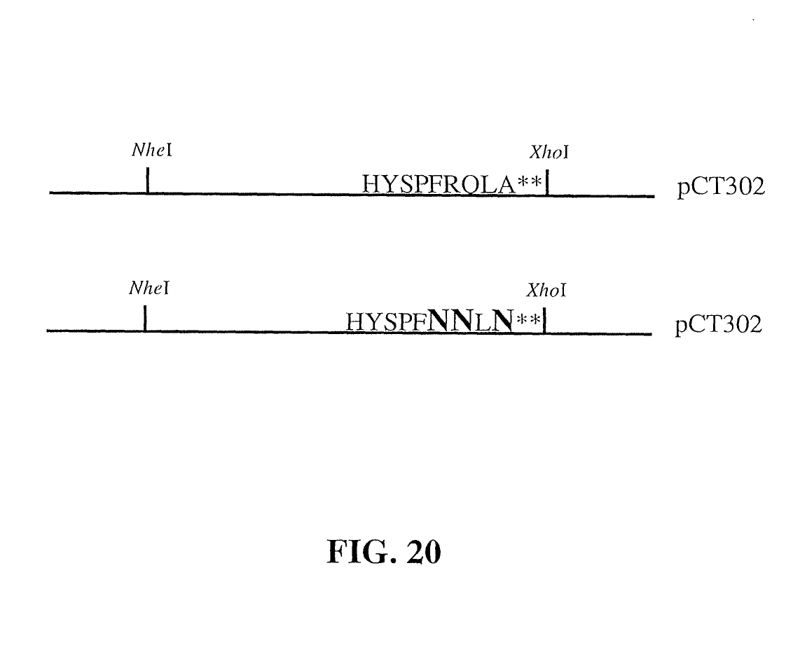

Another application of the yeast display to technology is to identify peptides that bind directly to MHC class I proteins. Serendipitously, we isolated a peptide that bound to the K^{b} molecule even though the peptide was fused to the C-terminus of the AGA2 protein. This finding was completely unexpected. Previously, only peptides of 8 to 10 amino acids had been shown to bind to class I MHC products. This restriction in length was thought to be due to the need for both the N- and C-termini to bind within pockets of the class I molecule, thereby contributing binding energy to the interaction. AGA2-fusion peptides were isolated by screening with a dEV8/K^{b} tetramer. A carboxy terminal peptide sequence HYSPFRQLA (SEQ ID NO:37) that had K^{b}-binding consensus anchor residues at positions P5 and P8 was isolated (FIG. 18). Yeast displaying the AGA2-fusion with the sequence HYSPFRQLA (SEQ ID NO:37) at the C-terminus bound to dEV8/K^{b} tetramers (FIG. 19). This binding was a consequence of the relative instability of dEV8 binding to K^{b}. This instability apparently allows the peptide dEV8 to dissociate, and the surface-bound AGA2-fusion peptide binds to the free K^{b}. Without wishing to be bound by any particular theory, we believe that the proline at position P4 allows the peptide to exit the K^{b}-site at the residue amino terminal to the P5 anchor. This is supported by the finding that an alanine substitution mutation at this position has significantly reduced binding (FIG. 19). These findings show that it is possible to create a library of fused peptides (to AGA2) and that this library can be screened for K^{b}-binding (FIG. 20). Thus, the yeast display system can be used to identify novel class I-binding peptides.

In order to provide a clear and consistent understanding of the specification and claims, including the scope to be given to such terms, the following definitions are provided.

A coding sequence is the part of a gene or cDNA which codes for the amino acid sequence of a protein, or for a functional RNA such as a tRNA or rRNA.

Complement or complementary sequence means a sequence of nucleotides which forms a hydrogen-bonded duplex with another sequence of nucleotides according to Watson-Crick base-pairing rules. For example, the complementary base sequence for 5′-AAGGCT-3′ is 3′-TTCCGA-5′.

Downstream means on the 3′ side of any site in DNA or RNA.

Expression refers to the transcription of a gene into structural RNA (rRNA, tRNA) or messenger RNA (mRNA) and subsequent translation of a mRNA into a protein.

An amino acid sequence that is functionally equivalent to a specifically exemplified MHC protein sequence is an amino acid sequence that has been modified by single or multiple amino acid substitutions, by addition and/or deletion of amino acids, or where one or more amino acids have been chemically modified, but which nevertheless retains the binding specificity and high affinity binding activity of a cell-bound or a soluble MHC protein of the present invention. Functionally equivalent nucleotide sequences are those that encode polypeptides having substantially the same biological activity as a specifically exemplified cell-bound or soluble MHC protein. In the context of the present invention, a soluble MHC protein is lacks the portions of a native cell-bound MHC and is stable in solution (i.e., it does not generally aggregate in solution when handled as described herein and under standard conditions for protein solutions).

Two nucleic acid sequences are heterologous to one another if the sequences are derived from separate organisms, whether or not such organisms are of different species, as long as the sequences do not naturally occur together in the same arrangement in the same organism.

Homology refers to the extent of identity between two nucleotide or amino acid sequences.

Isolated means altered by the hand of man from the natural state. If an “isolated” composition or substance occurs in nature, it has been changed or removed from its original environment, or both. For example, a polynucleotide or a polypeptide naturally present in a living animal is not isolated, but the same polynucleotide or polypeptide separated from the coexisting materials of its natural state is isolated, as the term is employed herein.

A linker region is an amino acid sequence that operably links two functional or structural domains of a protein.

A nucleic acid construct is a nucleic acid molecule which is isolated from a naturally occurring gene or which has been modified to contain segments of nucleic acid which are combined and juxtaposed in a manner which would not otherwise exist in nature.

Nucleic acid molecule means a single- or double-stranded linear polynucleotide containing either deoxyribonucleotides or ribonucleotides that are linked by 3′-5′-phosphodiester bonds.

Two DNA sequences are operably linked if the nature of the linkage does not interfere with the ability of the sequences to effect their normal functions relative to each other. For instance, a promoter region would be operably linked to a coding sequence if the promoter were capable of effecting transcription of that coding sequence.

A polypeptide is a linear polymer of amino acids that are linked by peptide bonds.

Promoter means a cis-acting DNA sequence, generally 80-120 base pairs long and located upstream of the initiation site of a gene, to which RNA polymerase binds and initiates correct transcription. There can be associated additional transcription regulatory sequences which provide on/off regulation of transcription and/or which enhance (increase) expression of the downstream coding sequence.

A recombinant nucleic acid molecule, for instance a recombinant DNA molecule, is a novel nucleic acid sequence formed in vitro through the ligation of two or more nonhomologous DNA molecules (for example a recombinant plasmid containing one or more inserts of foreign DNA cloned into at least one cloning site), through PCR technology or by directed homologous recombination, e.g. by co-transformation of two or more DNA molecules having at least regions of limited sequence identity to allow for homologous recombination with the transformed cell.

Transformation means the directed modification of the genome of a cell by the external application of purified recombinant DNA from another cell of different genotype, leading to its uptake and integration into the subject cell's genome. In bacteria, the recombinant DNA is not typically integrated into the bacterial chromosome, but instead replicates autonomously as a plasmid.

Upstream means on the 5′ side of any site in DNA or RNA.

A vector is a nucleic acid molecule that is able to replicate autonomously in a host cell and can accept foreign DNA. A vector carries at least one origin of replication functional in at least one type of cell, one or more unique recognition sites for restriction endonucleases which can be used for the insertion of foreign DNA, and usually selectable markers such as genes coding for antibiotic resistance, and often recognition sequences (e.g. promoter) for the expression of the inserted DNA. Common vectors include plasmid vectors and phage vectors. There can be more than one origin of replication to allow for replication and maintenance in more than one type of cell (e.g., separate origins of replication functional in yeast and Escherichia coli, respectively).

A virus infected cell is a cell in which a virus is replicating. Typically, a virus infected cell displays at least one antigen on its surface which is characteristic of the virus infection process. Such an antigen can be the target of recognition by the immune system and subsequent killing of the cell.

A pathogen infected cell is a human or animal cell in which an intracellular parasite (bacterial, fungal or protozoan) is surviving or reproducing. Typically a pathogen infected cell displays at least one antigen on its surface which is characteristic of the infection, and this antigen can be the target of immune recognition and targeting for killing of the infected cell.

The role of proteins encoded by the major histocompatibility complex (MHC proteins) have been known for nearly twenty years. MHC proteins are expressed by every individual, and they function as antigen-presenting molecules. Each MHC protein can bind to a variety of different small peptides (8 to 20 amino acids).

Recently it was demonstrated that a scTCR (Vβ-linker-Vα) could be displayed on the surface of yeast [Kieke et al. (1999) Proc. Natl. Acad. Sci. USA 96:5651-5656], in the yeast display system that has proven successful in antibody engineering [Boder and Wittrup (1997) Nat. Biotech. 15: 553-557.; Kieke et al. (1999) supra]. In addition, it was shown that mutations that increased the surface levels of the TCR also increased the sability of the TCR in solution [Shusta et al. (1999) J. Mol. Biol. 292:949-956]. Thus, yeast surface display can now be used to isolate proteins that exhibit greater stability.

It will be appreciated by those of skill in the art that, due to the degeneracy of the genetic code, numerous functionally equivalent nucleotide sequences encode the same amino acid sequence of an improved Class I MHC protein or class I MHC/peptide complex.

Additionally, those of skill in the art, through standard mutagenesis techniques, in conjunction with the antigen-finding activity assays described herein, can obtain altered MHC class I protein sequences and test them for the expression of polypeptides having particular binding activity. Useful mutagenesis techniques known in the art include, without limitation, oligonucleotide-directed mutagenesis, region-specific mutagenesis, linker-scanning mutagenesis, and site-directed mutagenesis by PCR [see e.g. Sambrook et al. (1989) vide infra, and Ausubel et al. (1999) vide infra].

In obtaining variant MHC coding sequences, those of ordinary skill in the art will recognize that MHC-derived proteins can be modified by certain amino acid substitutions, additions, deletions, and post-translational modifications, without loss or reduction of biological activity. In particular, it is well-known that conservative amino acid substitutions, that is, substitution of one amino acid for another amino acid of similar size, charge, polarity and conformation, are unlikely to significantly alter protein function. The 20 standard amino acids that are the constituents of proteins can be broadly categorized into four groups of conservative amino acids as follows: the nonpolar (hydrophobic) group includes alanine, isoleucine, leucine, methionine, phenylalanine, proline, tryptophan and valine; the polar (uncharged, neutral) group includes asparagine, cysteine, glutamine, glycine, serine, threonine and tyrosine; the positively charged (basic) group contains arginine, histidine and lysine; and the negatively charged (acidic) group contains aspartic acid and glutamic acid. Substitution in a protein of one amino acid for another within the same group is unlikely to have an adverse effect on the biological activity of the protein.

Homology between nucleotide sequences can be determined by DNA hybridization analysis, wherein the stability of the double-stranded DNA hybrid is dependent on the extent of base pairing that occurs. Conditions of high temperature and/or low salt content reduce the stability of the hybrid, and can be varied to prevent annealing of sequences having less than a selected degree of homology. For instance, for sequences with about 55% G-C content, hybridization and wash conditions of 40-50 C, 6× SSC (sodium chloride/sodium citrate buffer) and 0.1% SDS (sodium dodecyl sulfate) indicate about 60-70% homology, hybridization and wash conditions of 50-65 EC, 1× SSC and 0.1% SDS indicate about 82-97% homology, and hybridization and wash conditions of 52 C, 0.1× SSC and 0.1% SDS indicate about 99 -100% homology. A wide range of computer programs for comparing nucleotide and amino acid sequences (and measuring the degree of homology) are also available, and a list providing sources of both commercially available and free software is found in Ausubel et al. (1999). Readily available sequence comparison and multiple sequence alignment algorithms are, respectively, the Basic Local Alignment Search Tool (BLAST) (Altschul et al., 1997) and ClustalW programs. BLAST is available on the Internet at http://www.ncbi.nlm.nih.gov and a version of ClustalW is available athttp://www2.ebi.ac.uk.

Industrial strains of microorganisms (e.g., Aspergillus niger, Aspergillus ficuum, Aspergillus awamori, Aspergillus oryzae, Trichoderma reesei, Mucor miehei, Kluyveromyces lactis, Pichiapastoris, Saccharomyces cerevisiae, Escherichia coli, Bacillus subtilis or Bacillus licheniformis) or plant species (e.g., canola, soybean, corn, potato, barley, rye, wheat) may be used as host cells for the recombinant production of the mutant MHC proteins of the present invention. As the first step in the heterologous expression of a high affinity MHC protein or soluble protein, an expression construct is assembled to include the MHC or soluble MHC coding sequence and control sequences such as promoters, enhancers and terminators. Other sequences such as signal sequences and selectable markers may also be included. To achieve extracellular expression of a soluble MHC polypeptide, the expression construct may include a secretory signal sequence. The signal sequence is not included on the expression construct if cytoplasmic expression is desired. The promoter and signal sequence are functional in the host cell and provide for expression and secretion of the MHC or soluble MHC protein. Transcriptional terminators are included to ensure efficient transcription. Ancillary sequences enhancing expression or protein purification may also be included in the expression construct.

Various promoters (transcriptional initiation regulatory region) may be used according to the invention. The selection of the appropriate promoter is dependent upon the proposed expression host. Promoters from heterologous sources may be used as long as they are functional in the chosen host.

Promoter selection is also dependent upon the desired efficiency and level of peptide or protein production. Inducible promoters such tac are often employed in order to dramatically increase the level of protein expression E. coli. Overexpression of proteins may be harmful to the host cells. Consequently, host cell growth may be limited. The use of inducible promoter systems allows the host cells to be cultivated to acceptable densities prior to induction of gene expression, thereby facilitating higher product yields.

Various signal sequences may be used according to the invention. A signal sequence which is homologous to the MHC coding sequence may be used. Alternatively, a signal sequence which has been selected or designed for efficient secretion and processing in the expression host may also be used. For example, suitable signal sequence/host cell pairs include the B. subtilis sacB signal sequence for secretion in B. subtilis, and the Saccharomyces cerevisiae α-mating factor or P. pastoris acid phosphatase phoi signal sequences for P. pastoris secretion. The signal sequence may be joined directly through the sequence encoding the signal peptidase cleavage site to the protein coding sequence, or through a short nucleotide bridge consisting of usually fewer than ten codons, where the bridge ensures correct reading frame of the downstream TCR sequence.

Elements for enhancing transcription and translation have been identified for eukaryotic protein expression systems. For example, positioning the cauliflower mosaic virus (CaMV) promoter 1000 bp on either side of a heterologous promoter may elevate transcriptional levels by 10- to 400-fold in plant cells. The expression construct should also include the appropriate translational initiation sequences. Modification of the expression construct to include a Kozak consensus sequence for proper translational initiation may increase the level of translation by 10 fold.

A selective marker is often employed, which may be part of the expression construct or separate from it (e.g., carried by the expression vector), so that the marker may integrate at a site different from the gene of interest. Examples include markers that confer resistance to antibiotics (e.g., bla confers resistance to ampicillin for E. coli host cells, nptII confers kanamycin resistance to a wide variety of prokaryotic and eukaryotic cells) or that permit the host to grow on minimal medium (e.g., HIS4 enables P. pastoris or His-S. cerevisiae to grow in the absence of histidine). The selectable marker has its own transcriptional and translational initiation and termination regulatory regions to allow for independent expression of the marker. If antibiotic resistance is employed as a marker, the concentration of the antibiotic for selection will vary depending upon the antibiotic, generally ranging from 10 to 600 μg of the antibiotic/mL of medium.

The expression construct is assembled by employing known recombinant DNA techniques (Sambrook et al., 1989; Ausubel et al., 1999). Restriction enzyme digestion and ligation are the basic steps employed to join two fragments of DNA. The ends of the DNA fragment may require modification prior to ligation, and this may be accomplished by filling in overhangs, deleting terminal portions of the fragment(s) with nucleases (e.g., ExoIII), site directed mutagenesis, or by adding new base pairs by PCR. Polylinkers and adaptors may be employed to facilitate joining of selected fragments. The expression construct is typically assembled in stages employing rounds of restriction, ligation, and transformation of E. coli. Homlogons recombination strategies can be used with co-electroporation of linear DNAs into yeast (see FIG. 9). Numerous cloning vectors suitable for construction of the expression construct are known in the art (λZAP and pBLUESCRIPT SK-1, Stratagene, LaJolla, Calif.; pET, Novagen Inc., Madison, Wis.; cited in Ausubel et al., 1999) and the particular choice is not critical to the invention. The selection of cloning vector will be influenced by the gene transfer system selected for introduction of the expression construct into the host cell. At the end of each stage, the resulting construct may be analyzed by restriction, DNA sequence, hybridization and PCR analyses.

The expression construct may be transformed into the host as the cloning vector construct, either linear or circular, or may be removed from the cloning vector and used as is or introduced onto a delivery vector. The delivery vector facilitates the introduction and maintenance of the expression construct in the selected host cell type. The expression construct is introduced into the host cells by any of a number of known gene transfer systems (e.g., natural competence, chemically mediated transformation, protoplast transformation, electroporation, biolistic transformation, transfection, or conjugation) (Ausubel et al., 1999; Sambrook et al., 1989). The gene transfer system selected depends upon the host cells and vector systems used.

For instance, the expression construct can be introduced into S. cerevisiae cells by protoplast transformation or electroporation. Electroporation of S. cerevisiae is readily accomplished, and yields transformation efficiencies comparable to spheroplast transformation.

Monoclonal or polyclonal antibodies, preferably monoclonal, specifically reacting with a protein of interest may be made by methods known in the art. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York; and Ausubel et al. (1999) Current Protocols in Molecular Biology, John Wiley & Sons, Inc., New York.

MHC/peptide complexes of improved stability in cell-bound or soluble form which are characteristic of a particular neoplastic condition (cancer, tumor, or the like) or a particular virus infected cell or pathogen infected cell are useful, for example, as agonists of the immune system so that the neoplastic cell, virus infected cell or pathogen infected cell is more efficiently targeted for removal by T cells of the immune system. The improved MHC/p complexes can be labeled by joining, either covalently or noncovalently, a substance which provides a detectable signal. Suitable labels include but are not limited to radionuclides, enzymes, substrates, cofactors, inhibitors, fluorescent agents, chemiluminescent agents, magnetic particles and the like. Additionally the MHC protein of the present invention can be coupled to a ligand for a second binding molecules: for example, the MHC protein can be biotinylated. Detection of the MHC protein or complex can be effected by binding of a detectable streptavidin (a streptavidin to which a fluorescent, radioactive, chemiluminescent, or other detectable molecule is attached or to which an enzyme for which there is a chromophoric substrate available). United States patents describing the use of such labels and/or toxic compounds to be covalently bound to the scTCR protein include but are not limited to U.S. Pat. Nos. 3,817,837; 3,850,752; 3,927,193; 3,939,350; 3,996,345; 4,277,437; 4,275,149; 4,331,647; 4,348,376; 4,361,544; 4,468,457; 4,444,744; 4,640,561; 4,366,241; RE 35,500; 5,299,253; 5,101,827; 5,059,413. Labeled MHC proteins or complexes can be detected using a monitoring device or method appropriate to the label used. Fluorescence microscopy or fluorescence activated cell sorting can be used where the label is a fluorescent moiety, and where the label is a radionuclide, gamma counting, autoradiography or liquid scintillation counting, for example, can be used with the proviso that the method is appropriate to the sample being analyzed and the radionuclide used. In addition, there can be secondary detection molecules or particle employed where there is a detectable molecule or particle which recognized the portion of the MHC protein which is not part of the binding site for the cognate TCR or other ligand or other ligand in the absence of a component as noted herein. The art knows useful compounds for diagnostic imaging in situ; see, e.g., U.S. Pat. No. 5,101,827; 5,059,413. Radionuclides useful for therapy and/or imaging in vivo include ¹¹¹Indium, ⁹⁷Rubidium, ¹²⁵Iodine, ¹³¹Iodine, ¹²³Iodine, ⁶⁷Gallium, ⁹⁹Technetium. Toxins include diphtheria toxin, ricin and castor bean toxin, among others, with the proviso that once the TCR-toxin complex is bound to the cell, the toxic moiety is internalized so that it can exert its cytotoxic effect. Immunotoxin technology is well known to the art, and suitable toxic molecules include, without limitation, chemotherapeutic drugs such as vindesine, antifolates, e.g. methotrexate, cisplatin, mitomycin, anthrocyclines such as daunomycin, daunorubicin or adriamycin, and cytotoxic proteins such as ribosome inactivating proteins (e.g., diphtheria toxin, pokeweed antiviral protein, abrin, ricin, pseudomonas exotoxin A or their recombinant derivatives. See, generally, e.g., Olsnes and Pihl (1982) Pharmac. Ther. 25:355-381 and Monoclonal Antibodies for Cancer Detection and Therapy, Eds. Baldwin and Byers, pp. 159-179, Academic Press, 1985.

Table, high affinity MHC proteins specific for a particular ligand, e.g., a particular peptide, protein or cell type, are useful in diagnosing animals, including humans, believed to be suffering from a disease associated with the particular pMHC. The MHC molecules of the present invention are useful for detecting T cells that are specific for essentially any antigen including, but not limited to, those associated with a neoplastic condition, an abnormal protein, or an infection or infestation with a bacterium, a fungus, a virus, a protozoan, a yeast, a nematode or other parasite. Stable, high affinity MHC proteins specific for a particular ligand can also be used to induce the activity of T cells against antigens if desirable. For example, there are many peptides that have been associated with neoplastic cells, abnormal proteins, bacteria, fungi, a viruses, and protozoans whereby said peptides bind to a Class I MHC protein. Stable, high affinity MHC proteins in complex with these peptides could serve as vaccines against the diseases by inducing T cell activity.

The high affinity MHC compositions can be formulated by any of the means known in the art. They can be typically prepared as injectables, especially for intravenous, intraperitoneal or synovial administration (with the route determined by the particular disease) or as formulations for intranasal or oral administration, either as liquid solutions or suspensions. Solid forms suitable for solution in, or suspension in, liquid prior to injection or other administration may also be prepared. The preparation may also, for example, be emulsified, or the protein(s)/peptide(s) encapsulated in liposomes.

The active ingredients are often mixed with excipients or carriers which are pharmaceutically acceptable and compatible with the active ingredient. Suitable excipients include but are not limited to water, saline, dextrose, glycerol, ethanol, or the like and combinations thereof. The concentration of the MHC protein in injectable, aerosol or nasal formulations is usually in the range of 0.05 to 5 mg/ml. Similar dosages can be administered to other mucosal surfaces.

In addition, if desired, vaccines may contain minor amounts of auxiliary substances such as wetting or emulsifying agents, pH buffering agents, and/or adjuvants which enhance the effectiveness of the vaccine. Examples of adjuvants which may be effective include but are not limited to: aluminum hydroxide; N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine -2-(1′-2′-dipalmitoyl-sn-glycero-3hydroxyphosphoryloxy) -ethylamine (CGP 19835A, referred to as MTP-PE); and RIBI, which contains three components extracted from bacteria: monophosphoryl lipid A, trehalose dimycolate and cell wall skeleton (MPL+TDM+CWS) in a 2% squalene/Tween 80 emulsion. Such additional formulations and modes of administration as are known in the art may also be used.

The stable high affinity MHC proteins of the present invention and/or pMHC-binding fragments having primary structure similar (more than 90% identity) to the high affinity MHC proteins and which maintain the high affinity for the cognate ligand may be formulated into vaccines as neutral or salt forms. Pharmaceutically acceptable salts include but are not limited to the acid addition salts (formed with free amino groups of the peptide) which are formed with inorganic acids, e.g., hydrochloric acid or phosphoric acids; and organic acids, e.g., acetic, oxalic, tartaric, or maleic acid. Salts formed with the free carboxyl groups may also be derived from inorganic bases, e.g., sodium, potassium, ammonium, calcium, or ferric hydroxides, and organic bases, e.g., isopropylamine, trimethylamine, 2-ethylamino-ethanol, histidine, and procaine. Alternatively, these stable high affinity MHC proteins can be used as antagonists of an interaction between endogenous MHC proteins of similar specificity and the cognate TCR cells.

High affinity MHC proteins or complexes for therapeutic use, e.g., those conjugated to cytotoxic compounds are administered in a manner compatible with the dosage formulation, and in such amount and manner as are prophylactically and/or therapeutically effective, according to what is known to the art. The quantity to be administered, which is generally in the range of about 100 to 20,000 μg of protein per dose, more generally in the range of about 1000 to 10,000 μg of protein per dose. Similar compositions can be administered in similar ways using labeled high affinity MHC proteins for use in imaging, for example, to detect deleterious cytotoxic T cells that are involved in autoimmune attacks and containing the cognate pMHCs. Precise amounts of the active ingredient required to be administered may depend on the judgment of the physician or veterinarian and may be peculiar to each individual, but such a determination is well within the skill of such a practitioner.

The vaccine or other immunogenic composition may be given in a single dose; two dose schedule, for example two to eight weeks apart; or a multiple dose schedule. A multiple dose schedule is one in which a primary course of vaccination may include 1 to 10 or more separate doses, followed by other doses administered at subsequent time intervals as required to maintain and/or reinforce the immune response, e.g., at 1 to 4 months for a second dose, and if needed, a subsequent dose(s) after several months. Humans (or other animals) immunized with the retrovirus-like particles of the present invention are protected from infection by the cognate retrovirus.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzmol. 218, Part I; Wu (ed.) (1979) Meth Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) NucleicAcid Hybridization, IRL Press, Oxford, UK; and Setlow and Hollaender (1979) Genetic Engineering: Principles and Methods, Vols. 1-4, Plenum Press, New York. Abbreviations and nomenclature, where employed, are deemed standard in the field and commonly used in professional journals such as those cited herein.

All references cited in the present application are incorporated by reference herein to supplement the disclosure and experimental procedures provided in the present Specification to the extent that there is no inconsistency with the present disclosure.

The following examples are provided for illustrative purposes, and are not intended to limit the scope of the invention as claimed herein. Any variations in the exemplified articles and/or methods which occur to the skilled artisan are intended to fall within the scope of the present invention.

### EXAMPLES

### Example 1

### Fusion of the MHC α Chain to the β2m in the Yeast Display Vector pCT302

The mouse MHC K^{b} α chain was fused to the mouse β2m [Mottez et al. (1995) J. Exp. Med. 181(2), 493-502]. The K^{b} gene was PCR amplified using primers with Nhe I and Afl II restriction sites. The 5′ primer contained a 30 bp linker upstream of the K^{b} gene and the 3′ primer contained a 45 bp connecting linker (underlined) downstream of the gene (5′ CAA TGG CTA GCG GTG GAC TTA AGG GTG GAC CAG GTG GAG GTT CAG GAG GTG GAG GCC CAC ACT CGC TGA GGT ATT TCG T 3′, SEQ ID NO:7; and 5′ TGA ACC TCC GCC TCC TGA TCC ACC GCC ACC TGA ACC TAT TCC ACC CTC CCA TCT CAG GGT GAG GGG CTC AGG 3′, SEQ ID NO. 8). The β2m gene was PCR amplified with the 5′ primer containing the overlapping 45 bp linker (underlined) upstream and the 3′ primer containing a c-myc epitope tag and unique Xho I site downstream of the β2m (5′ GGT GGA ATA GGT TCA GGT GGC GGT GGA TCA GGA GGC GGA GGT TCA ATC CAG AAA ACC CCT CAA ATT CAA GTA T 3′, SEQ ID NO:9, and 5 ′GTT CCC TCG AGC TAT TAC AAG TCT TCT TCA GAA ATA AGC TTT TGT TCC ATG TCT CGA TCC CAG TAG ACG GT 3′, SEQ ID NO:10). Using PCR “sewing” of the overlapping linker [Davis et al. (1992) Biotechnology 9(2), 165-169], an amplified K^{b}/β2m fusion was generated using both the K^{b} and β2m PCR products, primers 1 and 4 and TaqPlus Precision PCR System (Stratagene, La Jolla, Calif.). The L^{d}/β2m MHC gene was fused and PCR amplified in an analogous manner. The K^{b}/β2m and L^{d}/β2m PCR products were digested with Nhe I/Xho I and ligated into the yeast surface display vector pCT302 containing a nine-residue epitope tag (HA) and the AGA2 open reading frame downstream of the inducible GALL promoter [Boder and Wittrup (1997) supra].

### Example 2

### Peptide/MHC Construction

The AGA2 gene was cloned at the COOH terminus of the peptide/K^{b}/β2m gene in order to allow a free NH₂ terminus of the peptide. First, the K^{b}/β2m signal sequence was PCR amplified with EcoRI and Nhe I restriction sites upstream and downstream of the signal sequence (SS) (5′ TCT CAA GAA TTC TAC TTC ATA CAT TTT 3′, SEQ ID NO:11; and

5′ GTA TCT GCT AGC TGC TAA AAC TGA AGC 3′, SEQ ID NO:12). The SS PCR product was digested Nhe I/Eco RI and ligated into the Nhe I/Eco RI digested pCT302 vector. The resulting plasmid contained the SS but not the AGA2 gene. Secondly, a SIYRYYGL (SEQ ID NO:1) (SIYR)/K^{b}/β2m PCR product was generated with a SIYR encoded primer (underlined) (5′ ATA CTA GCT AGC TTG GAT AAA AGG TCT ATT TAT AGA TAT TAT GGT TTG CTT AAG GGT GGA CCA GGT GGA GGT 3′, SEQ ID NO:13; and 5′ CAA TCC AGA TCT TTA CTA ATG CAA GTC TTC TTC AGA AAT AAG 3′, SEQ ID

NO:14). Nhe I and Nde I restriction sites are located in the upstream and downstream primers respectively. The SIYR/K^{b}/β2m PCR product was digested with Nhe I and Nde I and ligated into the Nhe I/Nde I digested pCT302 SS vector (now called SS-SIYR K^{b} pCT302). Finally, AGA2 was PCR amplified (5′ GGA TAT CAT ATG CAG GAA CTG ACA ACT ATA3′, SEQ ID NO:15, and 5′ ATT TGC AGA TCT CGA GTT ACT AAG CGT AGT CTG GAA CGT CGT A 3′, SEQ ID NO:16), digested with Nde I and Xho I and ligated into the Nde I/Xhol digested SS-SIYR K^{b} pCT302. The resulting construct contained the following order of genes in the pCT302 backbone: SS-SIYR K^{b}/β2m-AGA2.

### Example 3

### OVA and dEV8 Peptide Loading

The sense and anti-sense oligonucleotide sequences for both OVA (SIINFEKL, SEQ ID NO:2) and dEV8 (EQYKFYSV, SEQ ID NO:5) with a Nhe I and Afl II site upstream and downstream respectively were each phosphorylated with T4 DNA kinase (OVA 5° CT AGC TTG GAT AAA AGG AGC ATC ATC AAT TTT GAA AAG CTT C3′, SEQ ID NO:17; and 5′TT AAG AAG CTT TTC AAA ATT GAT GAT GCT CCT TTT ATC CAA G3′, SEQ ID NO:18; dEV8 5° CT AGC TTG GAT AAA AGG GAA CAA TAC AAA TTC TAC TCA GTT C3′, SEQ ID NO:19; and 5′TT AAG AAC TGA GTA GAA TTT GTA TTG TTC CCT TTT ATC CAA G3′, SEQ ID NO:20). The sense and anti-sense phosphorylated oligonucleotides were mixed (400 pmol), heated at 100° C. for 1 min and cooled slowly. Phosphorylated peptide cassettes were ligated into Nhe I/Afl II digested SIYRK^{b}/β2m pCT302. Ligation reactions were transformed into DHIOB electrocompetent E. coli cells and plated on LB/amp and incubated for 15 hrs at 37 C. Transformants were screened, and positive clones were sequenced. The resulting plasmids contained OVA and dEV8 tethered to K^{b}/β2m.

### Example 4

### Transformation into Yeast

The resultant MHC nucleotide constructs were transformed by the lithium acetate (LiAc) transformation method [Geitz et al. (1995) Yeast 11, 355-360] into the S. cerevisiae strain BJ5465 (α ura3-52 trp1 leu2Δ1 his3Δ200pep4::HIS2prbΔ1.6 can1 GAL; Yeast Genetic Stock Center, Berkeley, Calif.) containing a chromosomally integrated AGA1 coding sequence expressed under the control of the GAL1 promoter (strain EBY100; Boder and Wittrup (1997) supra).

### Example 5

### Induction and Detection of MHC on the Yeast Surface

Yeast cells transformed with pCT302/MHC plasmid constructs were grown overnight at 30 C with shaking in 2 mL selective glucose medium SD-CAA (glucose 2 wt %, Difco yeast nitrogen base 0.67 wt %, casamino acids 0.5 wt %). After 18-24 hours, recombinant AGAL+AGA2-MHC I expression was induced at 20° C. with shaking in 5 mL selective galactose medium (SG-CAA, where 2% galactose replaces the glucose in SD-CAA). Cultures were harvested after 24-48 hours (1-2 doublings) by centrifugation, washed with PBS (10 mM NaPO₄, 150 mM NaCl, pH 7.3) containing 0.5% bovine serum albumin and incubated 45 minutes on ice with 25 μL of an anti-MHC antibody, anti-c-myc Mab 9E10 (1:100 dilution of raw ascites fluid; Berkeley Antibody Co., Richmond, Calif.), or anti-HA Mab 12CA5 (10 μg/ml. Boehringer Mannheim, Indianapolis, Ind.). Cells were washed with PBS and incubated 30 minutes on ice with either FITC-labeled F(ab′)₂ goat anti-mouse IgG (1:50; Kirkegaard and Perry Labs, Inc., Gaithersburg, Md.) or a streptavidin-phycoerythrin (SA-PE) conjugate (1:200; PharMingen, San Diego, Calif.). Labeled yeast cells were analyzed on a Coulter Epics XL flow cytometer. Data for 10,000-20,000 events were collected, and the population was gated according to light scatter (size) to prevent analysis of cell clumps.

### Example 6

### Random Mutagenesis of dEV8/K^{b}/β2m and SIYR/K^{b}/β2m

dEV8/K^{b}/β2m and SIYR/K^{b}/β2m genes were randomly mutagenized using a PCR error prone technique. dEV8/K^{b}/β2m and SIYR/K^{b}/β2m were PCR amplified using vector specific primers at least 50 bp upstream and downstream of each gene with PCR conditions that cause random mutations to be inserted by Taq polymerase (GIBCO/BRL, Invitrogen,Carelsbad, Calif.). At a Mn:Mg ratio of about 0.16: 1, the polymerase is more susceptible to inserting a random nucleotide during elongation. Using homologous recombination [Raymond et al. (1999) Biotechniques 26(1): 134-138, 140-141], the dEV8/K^{b}/β2m and SIYR/K^{b}/β2m pCT302 vectors digested with Nhe and NdeI were combined with the error prone PCR products and electroporated (Bio-Rad Gene-Pulser II, 1.5V, 25μF, 0.2cm gene pulser cuvettes) into 40 μl of electrocompetent S. cerevisiae cells (BJ5465, strain EBY100). The resulting transformations (separately for the two chimeric genes) are pooled, and a dilution is plated on SD-CAA plates. Plates are incubated at 30 C for 3 days, and the library size is tabulated.

### Example 7

### Cell Sorting

The yeast library was grown in SD-CAA (2% dextrose, 0.67% yeast nitrogen base, 1% Casamino acids (Difco, Detroit, Mich.)) at 30 C to an OD₆₀₀=4.0. To induce surface scTCR expression, yeast cells were pelleted by centrifugation, resuspended to an OD₆₀₀=1.0 in SG-CAA (2% galactose, 0.67% yeast nitrogen base, 1% casamino acids), and incubated at 20 C for 48 hr. In general, 10⁷ cells/tube were incubated on ice for 1 hr with 50 μl of biotinylated anti-K^{b} antibody (B.8.24.3) diluted in phosphate buffered saline (pH 7.4) supplemented with 0.5 mg/ml BSA (PBS-BSA). After incubation, cells were washed and labeled for 30 min with SA:PE in PBS-BSA. Yeast cells were then washed and resuspended in PBS-BSA immediately prior to sorting. Cells exhibiting the highest fluorescence were isolated by FACS sorting with a Coulter 753 bench. After isolation, sorted cells were expanded in SD-CAA and induced in SG-CAA for subsequent rounds of selection. Three sequential sorts were performed for each mutant preparation with increasingly dilute anti-K^{b}. The percentages of total cells isolated from each sort were 1.0, 0.25 and 0. 1%, respectively. Aliquots of the third sorts were plated on SD-CAA to isolate individual clones, which were then analyzed by flow cytometry using a Coulter Epics XL instrument. The clones with the highest fluorescence have their DNA rescued with the Zymoprep Yeast Plasmid Miniprep Kit (Zymogen Research, Orange, Calif.). The DNA is retransformed into DH10 Belectrocompetent E. coli, mini prepped and submitted for sequencing. The sequences are analyzed, and the mutations are located.

TABLE 1
Comparison of Properties of H-2K^{b} , H-2D^{b} and H-2 L^{d} Proteins

| | H-2K^{b} | H-2D^{b} | H-2L^{d} |
|---|---|---|---|
| Heavy chain-B2m | | | |
| vdw | 17 | 24 | 7 |
| H bonds | 7 | 13 | 10 |
| alpha 1/alpha2: B2m | | | |
| vdw | 13 | 16 | 4 |
| H bonds | 4 | 5 | 2 |
| alpha 3: B2m | | | |
| vdw | 4 | 8 | 3 |
| H bonds | 3 | 8 | 8 |

TABLE 2
Various Single-Chain Class I MHC Constructions
Cloned in pCT3O2 Yeast Display Vector
Nucleic Acid Sequence 1:
SS-AGA2-K^{b}/β2m-c-myc (SEQ ID NO:22)

ATGCAGTTACTTCGCTGTTTTTCAATATTTTCTGTTATTGCTTCAGTTTT
AGCACAGGAACTGACAACTATATGCGAGCAAATCCCCTCACCAACTTTAG
AATCGACGCCGTACTCTTTGTCAACGACTACTATTTTGGCCAACGGGAAG
GCAATGCAAGGAGTTTTTGAATATTACAAATCAGTAACGTTTGTCAGTAA
TTGCGGTTCTCACCCCTCAACAACTAGCAAAGGCAGCCCCATAAACACAC
AGTATGTTTTTAAGGACAATAGCTCGACGATTGAAGGTAGATACCCATAC
GACGTTCCAGACTACGCTCTGCAGGCTAGTGGTGGTGGTGGTTCTGGTGG
TGGTGGTTCTGGTGGTGGTGGTTCTGCTAGCGGTGGACTTAAGGGTGGAC
CAGGTGGAGGTTCAGGAGGTGGAGGCCCACACTCGCTGAGGTATTTCGTC
ACCGCCGTGTCCCGGCCCGGCCTCGGGGAGCCCCGGTACATGGAAGTCGG
CTACGTGGACGACACGGAGTTCGTGCGCTTCGACAGCGACGCGGAGAATC
CGAGATATGAGCCGCGGGCGCGGTGGATGGAGCAGGAGGGGCCCGAGTAT
TGGGAGCGGGAGACACAGAAAGCCAAGGGCAATGAGCAGAGTTTCCGAGT
GGACCTGAGGACCCTGCTCGGCTACTACAACCAGAGCAAGGGCGGCTCTC
ACACTATTCAGGTGATCTCTGGCTGTGAAGTGGGGTCCGACGGGCGACTC
CTCCGCGGGTACCAGCAGTACGCCTACGACGGCTGCGATTACATCGCCCT
GAACGAAGACCTGAAAACGTGGACGGCGGCGGACATGGCGGCGCTGATCA
CCAAACACAAGTGGGAGCAGGCTGGTGAAGCAGAGAGACTCAGGGCCTAC
CTGGAGGGCACGTGCGTGGAGTGGCTCCGCAGATACCTGAAGAACGGGAA
CGCGACGCTGCTGCGCACAGATTCCCCAAAGGCCCATGTGACCCATCACA
GCAGACCTGAAGATAAAGTCACCCTGAGGTGCTGGGCCCTGGGCTTCTAC
CCTGCTGACATCACCCTGACCTGGCAGTTGAATGGGGAGGAGCTGATCCA
GGACATGGAGCTTGTGGAGACCAGGCCTGCAGGGGATGGAACCTTCCAGA
AGTGGGCATCTGTGGTGGTGCCTCTTGGGAAGGAGCAGTATTACACATGC
CATGTGTACCATCAGGGGCTGCCTGAGCCCCTCACCCTGAGATGGGAGGG
TGGAATAGGTTCAGGTGGCGGTGGATCAGGAGGCGGAGGTTCAATCCAGA
AAACCCCTCAAATTCAAGTATACTCACGCCACCCACCGGAGAATGGGAAG
CCGAACATACTGAACTGCTACGTAACACAGTTCCACCCGCCTCACATTGA
AATCCAAATGCTGAAGAACGGGAAAAAAATTCCTAAAGTAGAGATGTCAG
ATATGTCCTTCAGCAAGGACTGGTCTTTCTATATCCTGGCTCACACTGAA
TTCACCCCCACTGAGACTGATACATACGCCTGCAGAGTTAAGCATGACAG
TATGGCCGAGCCCAAGACCGTCTACTGGGATCGAGACATGGAACAAAAGC
TTATTTCTGAAGAAGACTTGTAATAGCTCGAG

Amino Acid Sequence 1:
SS-AGA2-K^{b}/β2m-c-myc (SEQ ID NO:23)

MQLLRCFSIFSVIASVLAQELTTICEQIPSPTLESTPYSLSTTTILANGK
AMQGVFEYYKSVTFVSNCGSHPSTTSKGSPINTQYVFKDNSSTIEGRYPY
DVPDYALQASGGGGSGGGGSGGGGSASGGLKGGPGGGSGGGGPHSLRYFV
TAVSRPGLGEPRYMEVGYVDDTEFVRFDSDAENPRYEPRARWMEQEGPEY
WERETQKAKGNEQSFRVDLRTLLGYYNQSKGGSHTIQVISGCEVGSDGRL
LRGYQQYAYDGCDYIALNEDLKTWTAADMAALITKHKWEQAGEAERLRAY
LEGTCVEWLRRYLKNGNATLLRTDSPKAHVTHHSRPEDKVTLRCWALGFY
PADITLTWQLNGEELIQDMELVETRPAGDGTFQKWASVVVPLGKEQYYTC
HVYHQGLPEPLTLRWEGGIGSGGGGSGGGGSIQKTPQIQVYSRHPPENGK
PNILNCYVTQFHPPHIEIQMLKNGKKIPKVEMSDMSFSKDWSFYILAHTE
FTPTETDTYACRVKHDSMAEPKTVYWDRDMEQKLISEEDL**LE

Nucleic Acid Sequence 2:
SS-SIYR/K^{b}/β2m-c-myc-AGA2 (SEQ ID NO:24)

GAATTCTACTTCATACATTTTCAATTAAGATGCAGTTACTTCGCTGTTTT
TCAATATTTTCTGTTATTGCTTCAGTTTTAGCAGCTAGCTTGGATAAAAG
ATCTATTTATAGATATTATGGTTTGCTTAAGGGTGGACCAGGTGGAGGTT
CAGGAGGTGGAGGCCCACACTCGCTGAGGTATTTCGTCACCGCCGTGTCC
CGGCCCGGCCTCGGGGAGCCCCGGTACATGGAAGTCGGCTACGTGGACGA
CACGGAGTTCGTGCGCTTCGACAGCGACGCGGAGAATCCGAGATATGAGC
CGCGGGCGCGGTGGATGGAGCAGGAGGGGCCCGAGTATTGGGAGCGGGAG
ACACAGAAAGCCAAGGGCAATGAGCAGAGTTTCCGAGTGGACCTGAGGAC
CCTGCTCGGCTACTACAACCAGAGCAAGGGCGGCTCTCACACTATTCAGG
TGATCTCTGGCTGTGAAGTGGGGTCCGACGGGCGACTCCTCCGCGGGTAC
CAGCAGTACGCCTACGACGGCTGCGATTACATCGCCCTGAACGAAGACCT
GAAAACGTGGACGGCGGCGGACATGGCGGCGCTGATCACCAAACACAAGT
GGGAGCAGGCTGGTGAAGCAGAGAGACTCAGGGCCTACCTGGAGGGCACG
TGCGTGGAGTGGCTCCGCAGATACCTGAAGAACGGGAACGCGACGCTGCT
GCGCACAGATTCCCCAAAGGCCCATGTGACCCATCACAGCAGACCTGAAG
ATAAAGTCACCCTGAGGTGCTGGGCCCTGGGCTTCTACCCTGCTGACATC
ACCCTGACCTGGCAGTTGAATGGGGAGGAGCTGATCCAGGACATGGAGCT
TGTGGAGACCAGGCCTGCAGGGGATGGAACCTTCCAGAAGTGGGCATCTG
TGGTGGTGCCTCTTGGGAAGGAGCAGTATTACACATGCCATGTGTACCAT
CAGGGGCTGCCTGAGCCCCTCACCCTGAGATGGGAGGGTGGAATAGGTTC
AGGTGGCGGTGGATCAGGAGGCGGAGGTTCAATCCAGAAAACCCCTCAAA
TTCAAGTATACTCACGCCACCCACCGGAGAATGGGAAGCCGAACATACTG
AACTGCTACGTAACACAGTTCCACCCGCCTCACATTGAAATCCAAATGCT
GAAGAACGGGAAAAAAATTCCTAAAGTAGAGATGTCAGATATGTCCTTCA
GCAAGGACTGGTCTTTCTATATCCTGGCTCACACTGAATTCACCCCCACT
GAGACTGATACATACGCCTGCAGAGTTAAGCATGACAGTATGGCCGAGCC
CAAGACCGTCTACTGGGATCGAGACATGGAACAAAAGCTTATTTCTGAAG
AAGACTTGCATATGCAGGAACTGACAACTATATGCGAGCAAATCCCCTCA
CCAACTTTAGAATCGACGCCGTACTCTTTGTCAACGACTACTATTTTGGC
CAACGGGAAGGCAATGCAAGGAGTTTTTGAATATTACAAATCAGTAACGT
TTGTCAGTAATTGCGGTTCTCACCCCTCAACAACTAGCAAAGGCAGCCCC
ATAAACACACAGTATGTTTTTAAGGACAATAGCTCGACGATTGAAGGTAG
ATACCCATACGACGTTCCAGACTACGCTTAGTAACTCGAG

Amino Acid Sequence 2:
SS-SIYR/K^{b}/β2m-c-myc-AGA2 (SEQ ID NO:25)

ILLHTFSIKMQLLRCFSIFSVIASVLAASLDKRSIYRYYGLLKGGPGGGS
GGGGPHSLRYFVTAVSRPGLGEPRYMEVGYVDDTEFVRFDSDAENPRYEP
RARWMEQEGPEYWERETQKAKGNEQSFRVDLRTLLGYYNQSKGGSHTIQV
ISGCEVGSDGRLLRGYQQYAYDGCDYIALNEDLKTWTAADMAALITKHKW
EQAGEAERLRAYLEGTCVEWLRRYLKNGNATLLRTDSPKAHVTHHSRPED
KVTLRCWALGFYPADITLTWQLNGEELIQDMELVETRPAGDGTFQKWASV
VVPLGKEQYYTCHVYHQGLPEPLTLRWEGGIGSGGGGSGGGGSIQKTPQI
QVYSRHPPENGKPNILNCYVTQFHPPHIEIQMLKNGKKIPKVEMSDMSFS
KDWSFYILAHTEFTPTETDTYACRVKHDSMAEPKTVYWDRDMEQKLISEE
DLHMQELTTICEQIPSPTLESTPYSLSTTTILANGKAMQGVFEYYKSVTF
VSNCGSHPSTTSKGSPINTQYVFKDNSSTIEGRYPYDVPDYA

Nucleic Acid Sequence 3:
SS-dEV8/K^{b}/β2m-c-myc-AGA2 (SEQ ID NO:26)

GAATTCTACTTCATACATTTTCAATTAAGATGCAGTTACTTCGCTGTTTT
TCAATATTTTCTGTTATTGCTTCAGTTTTAGCAGCTAGCTTGGATAAAAG
AGAACAATACAAATTCTACTCAGTTCTTAAGGGTGGACCAGGTGGAGGTT
CAGGAGGTGGAGGCCCACACTCGCTGAGGTATTTCGTCACCGCCGTGTCC
CGGCCCGGCCTCGGGGAGCCCCGGTACATGGAAGTCGGCTACGTGGACGA
CACGGAGTTCGTGCGCTTCGACAGCGACGCGGAGAATCCGAGATATGAGC
CGCGGGCGCGGTGGATGGAGCAGGAGGGGCCCGAGTATTGGGAGCGGGAG
ACACAGAAAGCCAAGGGCAATGAGCAGAGTTTCCGAGTGGACCTGAGGAC
CCTGCTCGGCTACTACAACCAGAGCAAGGGCGGCTCTCACACTATTCAGG
TGATCTCTGGCTGTGAAGTGGGGTCCGACGGGCGACTCCTCCGCGGGTAC
CAGCAGTACGCCTACGACGGCTGCGATTACATCGCCCTGAACGAAGACCT
GAAAACGTGGACGGCGGCGGACATGGCGGCGCTGATCACCAAACACAAGT
GGGAGCAGGCTGGTGAAGCAGAGAGACTCAGGGCCTACCTGGAGGGCACG
TGCGTGGAGTGGCTCCGCAGATACCTGAAGAACGGGAACGCGACGCTGCT
GCGCACAGATTCCCCAAAGGCCCATGTGACCCATCACAGCAGACCTGAAG
ATAAAGTCACCCTGAGGTGCTGGGCCCTGGGCTTCTACCCTGCTGACATC
ACCCTGACCTGGCAGTTGAATGGGGAGGAGCTGATCCAGGACATGGAGCT
TGTGGAGACCAGGCCTGCAGGGGATGGAACCTTCCAGAAGTGGGCATCTG
TGGTGGTGCCTCTTGGGAAGGAGCAGTATTACACATGCCATGTGTACCAT
CAGGGGCTGCCTGAGCCCCTCACCCTGAGATGGGAGGGTGGAATAGGTTC
AGGTGGCGGTGGATCAGGAGGCGGAGGTTCAATCCAGAAAACCCCTCAAA
TTCAAGTATACTCACGCCACCCACCGGAGAATGGGAAGCCGAACATACTG
AACTGCTACGTAACACAGTTCCACCCGCCTCACATTGAAATCCAAATGCT
GAAGAAGAACGGAAAAATTCCTAAAGTAGAGATGTCAGATATGTCCTTCA
GCAAGGACTGGTCTTTCTATATCCTGGCTCACACTGAATTCACCCCCACT
GAGACTGATACATACGCCTGCAGAGTTAAGCATGACAGTATGGCCGAGCC
CAAGACCGTCTACTGGGATCGAGACATGGAACAAAAGCTTATTTCTGAAG
AAGACTTGCATATGCAGGAACTGACAACTATATGCGAGCAAATCCCCTCA
CCAACTTTAGAATCGACGCCGTACTCTTTGTCAACGACTACTATTTTGGC
CAACGGGAAGGCAATGCAAGGAGTTTTTGAATATTACAAATCAGTAACGT
TTGTCAGTAATTGCGGTTCTCACCCCTCAACAACTAGCAAAGGCAGCCCC
ATAAACACACAGTATGTTTTTAAGGACAATAGCTCGACGATTGAAGGTAG
ATACCCATACGACGTTCCAGACTACGCTTAGTAACTCGAG

Amino Acid Sequence 3:
SS-dEV8/K^{b}/β2m-c-myc-AGA2 (SEQ ID NO:27)

ILLHTFSIKMQLLRCFSIFSVIASVLAASLDKREQYKFYSVLKGGPGGGS
GGGGPHSLRYFVTAVSRPGLGEPRYMEVGYVDDTEFVRFDSDAENPRYEP
RARWMEQEGPEYWERETQKAKGNEQSFRVDLRTLLGYYNQSKGGSHTIQV
ISGCEVGSDGRLLRGYQQYAYDGCDYIALNEDLKTWTAADMAALITKEKL
EGTCVEWLRRYLKNGNATLLRTDSPKAHVTHHSRPEDKVTLRCWALGFYP
ADITLTWQLNGEELIQDMELVETRPAGDGTFOKWASVVVPLGKEQYYTCH
VYHQGLPEPLTLRWEGGIGSGGGGSGGGGSIQKTPQIQWSRHPPENGKPN
ILNCYVTQFHPPHIEIQMLKNGKKIPKVEMSDMSFSKDWSFYILATEFTP
TETDTYACRVKHDSMAEPKTVYWDRDMEQKLISEEDLHQELTTICEQIPS
PTLESTPYSLSTTTILANGKMQGVFEYYKSVTFVSNCGSHPSTTSKGSPI
NTQYVFKDNSSTIEGRYPYDVPDYA

Nucleic Acid Sequence 4:
SS-OVA/K^{b}/β2m-c-myc-AGA2 (SEQ ID NO:28)

GAATTCTACTTCATACATTTTCAATTAAGATGCAGTTACTTCGCTGTTTT
TCAATATTTTCTGTTATTGCTTCAGTTTTAGCAGCTAGCTTGGATAAAAG
GAGCATCATCAATTTTGAAAAGCTTCTTAAGGGTGGACCAGGTGGAGGTT
CAGGAGGTGGAGGCCCACACTCGCTGAGGTATTTCGTCACCGCCGTGTCC
CGGCCCGGCCTCGGGGAGCCCCGGTACATGGAAGTCGGCTACGTGGACGA
CACGGAGTTCGTGCGCTTCGACAGCGACGCGGAGAATCCGAGATATGAGC
CGCGGGCGCGGTGGATGGAGCAGGAGGGGCCCGAGTATTGGGAGCGGGAG
ACACAGAAAGCCAAGGGCAATGAGCAGAGTTTCCGAGTGGACCTGAGGAC
CCTGCTCGGCTACTACAACCAGAGCAAGGGCGGCTCTCACACTATTCAGG
TGATCTCTGGCTGTGAAGTGGGGTCCGACGGGCGACTCCTCCGCGGGTAC
CAGCAGTACGCCTACGACGGCTGCGATTACATCGCCCTGAACGAAGACCT
GAAAACGTGGACGGCGGCGGACATGGCGGCGCTGATCACCAAACACAAGT
GGGAGCAGGCTGGTGAAGCAGAGAGACTCAGGGCCTACCTGGAGGGCACG
TGCGTGGAGTGGCTCCGCAGATACCTGAAGAACGGGAACGCGACGCTGCT
GCGCACAGATTCCCCAAAGGCCCATGTGACCCATCACAGCAGACCTGAAG
ATAAAGTCACCCTGAGGTGCTGGGCCCTGGGCTTCTACCCTGCTGACATC
ACCCTGACCTGGCAGTTGAATGGGGAGGAGCTGATCCAGGACATGGAGCT
TGTGGAGACCAGGCCTGCAGGGGATGGAACCTTCCAGAAGTGGGCATCTG
TGGTGGTGCCTCTTGGGAAGGAGCAGTATTACACATGCCATGTGTACCAT
CAGGGGCTGCCTGAGCCCCTCACCCTGAGATGGGAGGGTGGAATAGGTTC
AGGTGGCGGTGGATCAGGAGGCGGAGGTTCAATCCAGAACCCCTCAARTT
CAAGTATACTCACGCCACCCACCGGAGAATGGGAAGCCGAACATACTGAA
CTGCTACGTAACACAGTTCCACCCGCCTCACATTGAAATCCAAATGCTGA
AGAACGGGAAAAAAATTCCTAAAGTAGAGATGTCAGATATGTCCTTCAGC
AAGGACTGGTCTTTCTATATCCTGGCTCACACTGAATTCACCCCCACTGA
GACTGATACATACGCCTGCAGAGTTAAGCATGACAGTATGGCCGAGCCCA
AGACCGTCTACTGGGATCGAGACATGGAACAAAAGCTTATTTCTGAAGAA
GACTTGCATATGCAGGAACTGACAACTATATGCGAGCAAATCCCCTCACC
AACTTTAGAATCGACGCCGTACTCTTTGTCAACGACTACTATTTTGGCCA
ACGGGAGGCAATGCAAGGAAGAATATTACAAATCAGTAACGTTTGTCAGT
AATTGCGGTTCTCACCCCTCAACAACTAGCAAAGGCAGCCCCATAAACAC
ACAGTATGTTTTTAAGGACAATAGCTCGACGATTGAAGGTAGATACCCAT
ACGACGTTCCAGACTACGCTTAGTAACTCGAG

Amino Acid Sequence 4:
SS-OVA/K^{b}/β2m-c-myc-AGA2 (SEQ ID NO:29)

ILLHTFSIKMQLLRCFSIFSVIASVLAASLDKRSIINFEKLLKGGPGGGS
GGGGPHSLRYFVTAVSRPGLGEPRYMEVGYVDDTEFVRFDSDAENPRYEP
RARWMEQEGPEYWERETQKAKGNEQSFRVDLRTLLGYYNQSKGGSHTIQV
ISGCEVGSDGRLLRGYQQYAYDGCDYIALNEDLKTWTAADMAALITKHKW
EQAGEAERLRAYLEGTCVEWLRRYLKNGNATLLRTDSPKAHVTHHSRPED
KVTLRCWALGFYPADITLTWQLNGEELIQDMELVETRPAGDGTFQKWASV
VVPLGKEQYYTCHVYHQGLPEPLTLRWEGGIGSGGGGSGGGGSIQKTPQI
QVYSRHPPENGKPNILNCYVTQFHPPHIEIQMLKNGKKIPKVEMSDMSFS
KDWSFYILAHTEFTPETDTYACRVKHDSMAEPKTVYWDRDMEQKLISEED
LHMQELTTICEQIPSPTLESTPYSLSTTTILANGKAMQGVFEYYKSVTFV
SNCGSHPSTTSKGSPINTQYVFKDNSSTIEGRYPYDVPDYA

Nucleic Acid Sequence 5:
SS-AGA2-L^{d}/β2m-c-myc (SEQ ID NO:30)

ATGCAGTTACTTCGCTGTTTTTCAATATTTTCTGTTATTGCTTCAGTTTT
AGCACAGGACTGACAACTATATGCGAGCAATCCCCTCACCTGGACGCCGT
ACTCTTTGTCAACGACTACTATTTTGGCCAACGGGAAGGCAATGCAAGGA
GTTTTTGAATATTACAAATCAGTAACGTTTGTCAGTTTGCGGTTCTCACC
CCTCAACAACTAGCAAAGGCAGCCCCATAAACACACAGTATGTTTTTGGA
CAATAGCTCGACGATTGAAGGTAGATACCCATACGACGTTCCAGACTACG
CTCTGCAGGCTAGTGGTGGTGGTGGTTCGGTGGTGGTGGTTCTGGTGGTG
GTGGTTCTGCTAGCGGTGGACTTAAGGGTGGACCAGGTGGAGGTTCAGGA
GGTGGAGGCCCACACTCGATGCGGTATTTCGAGACCGCGGTGTCCCGGCG
CGGCCTCGGGGAGCCCCGGTACATCTCTGTCGGCTATGTGAACGACAAGG
AGTTCGTGCGCTTCGACAGCGACGCGGAGAATCCGAGATATGAGCCGAGG
GCGCCGTGGATGGAGCAGGAGGGGCCGGAGTATTGGGAGCGGATCACGCA
GATCGCCAAGGGCCAGGAGCAGTGGTTCCGAGTGAACCTGAGGACCCTGC
TCGGCTACTACAACCAGAGCGCGGGCGGCACTCACACACTCCAGTGGATG
TACGGCTGTGACGTGGGGTCGGACGGGCGCCTCCTCCGCGGGTACGAGCA
GTTCGCCTACGACGGCTGCGATTACATCGCCCTGAACGAAGACCTGAAAA
CGTGGACGTTCGCGGACATGTCGTCGATGATCACCCGACGCAAGTGGGAG
CAGGCTGGTGCTGCAGAGTATTACAGGGCCTACCTGGAGGGCGAGTGCGT
GGAGTGGCTCCACAGATACCTGAAGAACGGGAATGCTACGCTGCTGCGCA
CAGATTCCCCAAAGGCACATGTGACCTATCACCCCAGATCTAAAGGTGAA
GTCACCCTGAGGTGCTGGGCCCTGGGCTTCTACCCTGCTGACATCACCCT
GACCTGGCAGTTGAATGGGGAGGAGCTGACCCAGGACATGGAGCTTGTGG
AGACCAGGCCTGCAGGGGATGGAACCTTCCAGAAGTGGGCATCTGTGGTG
GTGCCTCTTGGGAAGGAGCAGAATTACACATGCCGTGTGTACCATGAGGG
GCTGCCCCATCCCCTCACCCTGAGATGGGAGGGTGGAATAGGTTCAGGTG
GCGGTGGATCAGGAGGCGGAGGTTCAATCCAGAAAACCCCTCAAATTCAA
GTATACTCACGCCACCCACCGGAGAATGGGAAGCCGAACATACTGAACTG
CTACGTAACACAGTTCCACCCGCCTCACATTGAAATCCAAATGCTGAAGA
AGGGGAAAAAAATTCCTAAAGTAGAGATGTCAGATATGTCCTTCAGCAAG
GACTGGTCTTTCTATATCCTGGCTCACACTGAATTCACCCCCACTGAGAC
TGATACATACGCCTGCAGAGTTAAGCATGACAGTATGGCCGAGCCCAAGA
CCGTCTACTGGGATCGAGACATGGAACAAAAGCTTATTTCTGAAGAAGAC
TTGTAATAGCTCGAG

Amino Acid Sequence 5:
SS-AGA2-L^{d}/β2m-c-myc (SEQ ID NO:31)

MQLLRCFSIFSVIASVLAQELTTICEQIPSPTLESTPYSLSTTTILANGK
AMQGVFEYYKSVTFVSNCGSHPSTTSKGSPINTQYVFKDNSSTIEGRYPY
DVPDYALQASGGGGSGGGGSGGGGSASGGLKGGPGGGSGGGGPHSMRYFE
TAVSRRGLGEPRYISVGYVNDKEFVRFDSDAENPRYEPRAPWMEQEGPEY
WERITQIAKGQEQWFRVNLRTLLGYYNQSAGGTHTLQWMYGCDVGSDGRL
LRGYEQFAYDGCDYIALNEDLKTWTFADMSSMITRRKWEQAGAAEYYRAY
LEGECVEWLHRYLKNGNATLLRTDSPKAHVTYHPRSKGEVTLRCWALGFY
PADITLTWQLNGEELTQDMELVETRPAGDGTFQKWASVVVPLGKEQNYTC
RVYHEGLPHPLTLRWEGGIGSGGGGSGGGGSIQKTPQIQVYSRHPPENGK
PNILNCYVTQFHPPHIEIQMLKNGKKIPKVEMSDMSFSKDWSFYILAHTE
FTPTETDTYACRVKHDSMAEPKTVYWDRDMEQKLISEEDL

TABLE 3
Sequences of mutant clones isolated by sorting
from dEV8/Kb error-prone PCR library.
Nucleic Acid Sequence 1: dEP.1 (SEQ ID NO:32)

GAATTCTACTTCATACATTTTCAATTAAGATGCAGTTACTTCGCTGTTTT
TCAATATTTTCTGTTATTGCTTCAGTTTTAGCAGCTAGCTTGGATAAAAG
AGAACAATACAAATTCTACTCAGTTCTTAAGGGTGGACCAGGTGGAGGTT
CAGGAGGTGGAGGCCCACACTCGCTGAGGTATTTCGTCACCGCCGTGTCC
CGGCCCGGCCTCGGGGAGCCCCGGTACATGGAAGTCGGCTACGTGGACGA
CACGGAGTTCGTGCGCTTCGACAGCGACGCGGAGAATCCGAGATATGAGC
CGCGGGCGCGGTGGATGGAGCAGGAGGGGCCCGAGTATTGGGAGCGGGAG
ACACAGAAAGCCAAGGGCAATGAGCAGAGTTTCCGAGTGGACCTGAGGAC
CCTGCTCGGCTACTACAACCAGAGCAAGGGCGGCTCTCACACTATTCAGG
TGATCTCTGGCTGTGGAATGGGGTCCGACGGGCGACTCCTCCGCGGGTAC
CAGCAGTACGCCTACGACGGCTGCGATTACATCGCCCTGAACGAAGACCT
GAAAACGTGGACGGCGGCGGACATGGCGGCGCTGATCACCAAACACAAGT
GGGAGCAGGCTGGTGAAGCAGAGAGACTCAGGGCCTACCTGGAGGGCACG
TGCGTGGAGAGGCTCCGCAGATACCTGAAGAACGGGAACGCGACGCTGCT
GCGCACAGATTCCCCAAAGGCCCATGTGACCCATCACAGCAGACCTGAAG
ATAAAGTCACCCTGAGGTGCTGGGCCCTGGGCTTCTACCCTGCTGACATC
ACCCTGACCTGGCAGTTGAATGGGGAGGAGCT

Amino Acid Sequence 1: dEP.1 (SEQ ID NO:33)

ILLHTFSIKMQLLRCFSIFSVIASVLAASLDKREQYKFYSVLKGGPGGGS
GGGGPHSLRYFVTAVSRPGLGEPRYMEVGYVDDTEFVRFDSDAENPRYEP
RARWMEQEGPEYWERETQKAKGNEQSFRVDLRTLLGYYNQSKGGSHTIQV
ISGCEVGSDGRLLRGYQQYAYDGCDYIALNEDLKTWTAADMAALITKHKW
EQAGEAERLRAYLEGTCVERLRRYLKNGNATLLRTDSPKAHVTHHSRPED
KVTLRCWALGFYPADITLTWQLNGEELIQDMELVETRPAGDGTFQKWASV
VVPLGKEQYYTCHVYHQGLPEPLTLRWEGGIGSGGGGSGGGGSIQKTPQI
QVYSRHPPENGKPNILNCYVTQFHPPHIEIQMLKNGKKIPKVEMSDMSFS
KDWSFNILAHTEFTPTETDTYACRVKHDSMAEPKTVYWDRDMEQRLISEE
DLHMQELTTICEQIPSPTLESTPYSLSTTTILANGKAMQGVFEYYESVTF
VSNCGSHPSTTSKGSPINTQYVFKDNSSTIEGRYPYDVPDYA

Nucleic Acid Sequence 2: dEP.3 (SEQ ID NO:34)

GAATTCTACTTCATACATTTTCAATTAAGATGCAGTTACTTCGCTGTTTT
TCAATATTTTCTGTTATTGCTTCAGTTTTAGCAGCTAGCTTGGATAAAAG
AGAACAATACAAATTCTACTCAGTTCTTAAGGGTGGACCAGGTGGAGGTT
CAGGAGGTGGAGGCCCACACTCGCTGAGGTATTTCGTCACCGCCGTGTCC
CGGCCCGGCCTCGGGGAGCCCCGGTACATGGAAGTCGGCTACGTGGACGA
CACGGAGTTCGTGCGCTTCGACAGCGACGCGGAGAATCCGAGATATGAGC
CGCGGGCGCGGTGGATGGAGCAGGAGGGGCCCGAGTATTGGGAGCGGGAG
ACACAGAAAGCCAAGGGCAATGAGCAGAGTTTCCGAGTGGACCTGAGGAC
CCTGCTCGGCTACTACAACCAGAGCAAGGGCGGCTCTCACACTATTCAGG
TGATCTCTGGCTGTGAAGTGGGGTCCGACGGGCGACTCCTCCGCGGGTAC
CAGCAGTACGCCTACGACGGCTGCGATTACATCGCCCTGAACGAAGACCT
GAAAACGTGGACGGCGGCGGACATGGCGGCGCTGATCACCAAACACAAGT
GGGAGCAGGCTGGTGAAGCAGAGAGACTCAGGGCCTACCTGGAGGGCACG
TGCGTGGAGCGGCTCCGCAGATACCTGAAGAACGGGAACGCGACGCTGCT
GCGCACAGATTCCCCAAAGG

Amino Acid Sequence 2: dEP.3 (SEQ ID NO:35)

ILLHTFSIKMQLLRCFSIFSVIASVLAASLDKREQYKFYSVLKGGPGGGS
GGGGPHSLRYFVTAVSRPGLGEPRYMEVGYVDDTEFVRFDSDAENPRYEP
RARWMEQEGPEYWERETQKAKGNEQSFRVDLRTLLGYYNQSKGGSHTIQV
ISGCEVGSDGRLLRGYQQYAYDGCDYIALNEDLKTWTAADMAALITKHKW
EQAGEAERLRAYLEGTCVERLRRYLKNGNATLLRTDSPKAHVTHHSRPED
KVTLRCWALGFYPADITLTWQLNGEELIQDMELVETRPAGDGTFQKWASV
VVPLGKEQYYTCHVYHQGLPEPLTLRWEGGIGSGGGGSGGGGSIQKTPQI
QVYSRHPPENGKPNILNCYVTQFHPPHIEIQMLKNGKKIPKVEMSDMSFS
KDWSFYILAHTEFTPTETDTYACRVKHDSMAEPKTVYWDRDMEQKLISEE
DLHMQELTTICEQIPSPTLESTPYSLSTTTILANGKAMQGVFEYYKSVTF
VSNCGSHPSTTSKGSPINTQYVFKDNSSTIEGRYPYDVPDYA

Nucleic Acid Sequence 3: dEP.4 (SEQ ID NO:36)

GAATTCTACTTCATACATTTTCAATTAAGATGCAGTTACTTCGCTGTTTT
TCAATATTTTCTGTTATTGCTTCAGTTTTAGCAGCTAGCTTGGATAAAAG
AGAACAATACAAATTCTACTCAGTTCTTAAGGGTGGACCAGGTGGAGGTT
CAGGAGGTGGAGGCCCACACTCGCTGAGGTATTTCGTCACCGCCGTGTCC
CGGCCCGGCCTCGGGGAGCCCCGGTACATGGAAGTCGGCTACGTGGACGA
CACGGAGTTCGTGCGCTTCAACAGCGACGCGGAGAATCCGAGATATGAGC
CGCGGGCGCGGTGGATGGAGCAGGAGGGGCCCGAGTATTGGGAGCGGGAG
ACACAGAAAGGCAAGGGCAATGAGCAGAGTTTCCGAGTGGACCTGAGGAC
CCTGCTCGGCTACTACAACCAGAGCAAGGGCGGCTCTCACACTATTCAGG
TGATCTCTGGCTGTGAAGTGGGGTCCGACGGGCGACTCCTCCGCGGGTAC
CAGCAGTACGCCTACGACGGCTGCGATTACATCGCCCTGAACGAAGACCT
GAAAACGTGGACGGCGGCGGACATGGCGGCGCTGATCACCAAACACAAGT
GGGAGCAGGCTGGTGAAGCAGAGAGACTCAGGGCCTACCTGGAGGGCACG
TGCGTGGAGAGGCTCCGCAGATACCTGAAGAACGGGAACGCGACGCTGCT
GCGCACAGATTCCCCAAAGGCCCATGTGACCCATCACAGCAGACCTGAAG
ATAAAGTCACCCTGAGGTGCTGGGCCCTGGGCTTCTACCCTGCTGACATC
ACCCTGACCTGGCAGTTGAATGGGGAGGAGCTGATCCAGGACATGGAGCT
TGTGGAGACCAGGCCTGCAGGGGATGGAACCTTCCAGAAGTGGGCATCTG
TGGTGGTGCCTCTTGGGAAGGAGCAGTATTACACATGCCATGTGTACCAT
CAGGGGCTGCCTGAGCCCCTCACCCTGAGATGGGAGGGTGGAATAGGTTC
AGGTGGCGGTGGATCAGGAGGCGGAGGTTCAATCCAGAAAACCCCTCAAA
TTCAAGTATACTCACGCCACCCACCGGAGAATGGGAAGCCG

Amino Acid Sequence 3: dEP.4 (SEQ ID NO:37)

ILLHTFSIKMQLLRCFSIFSVIASVLAASLDKREQYKFYSVLKGGPGGGS
GGGGPHSLRYFVTAVSRPGLGEPRYMEVGVVDDTEFVRFNSDAENPRYEP
RARWMEQEGPEYWERETQKAKGNEQSFRVDLRTLLGYYNQSKGGSHTIQV
ISGCEVGSDGRLLRGYQQYAYDGCDYIALNEDLKTWTAADMAALITKHKW
EQAGEAERLRAYLEGTCVERLRRYLKNGNATLLRTDSPKAHVTHHSRPED
KVTLRCWALGFYPADITLTWQLNGEELIQDMELVETRPAGDGTFQKWASV
VVPLGKEQYYTCHVYHQGLPEPLTLRWEGGIGSGGGGSGGGGSIQKTPQI
QVYSRHPPENGKPNILNCYVTQFHPPHIEIQMLKNGKKIPKVEMSDMSFS
KDWSFNILAHTEFTPTETDTYACRVKHDSMAEPKTVYWDRDMEQRLISEE
DLHMQELTTICEQIPSPTLESTPYSLSTTTILANGKAMQGVFEYYKSVTF
VSNCGSHPSTTSKGSPINTQYVFKDNSSTIEGRYPYDVPDYA

### REFERENCES

Altschul, S. F. et al. (1997) Nucl. Acids Res. 25: 3389-3402.

Boder, E. T., and Wittrup, K. D. (1997) Nat. Biotech. 15, 553-557.

Bolin, D. R. et al. (2000) J. Med. Chem. 43(11), 2135-2148.

Bouvier, M., and Wiley, D. C. (1994) Science 265(5170), 398-402.

Brusic, V. et al. (1998. Bioinformatics 14(2), 121-130.

Carrasco-Marin, E. et al. (1996) J. Immunol. 156(2), 450-458.

Castano, A. R. et al. (1995) Science 269(5221), 223-226.

Cho, B. K. et al. (1998) J. Immunol. Methods 220(1-2), 179-188.

Crawford, F. et al. (1998) Immunity 8(6), 675-682.

Cresswell, P. (1994) Annu. Rev. Immunol. 12, 259-293.

Davenport, M. P. et al. (1996) Immunology 88(4), 482-486.

Davis, G. T. et al. (1991) Biotechnology 9(2), 165-169.

Engelhard, V. H. (1994) Curr. Op. Immunol. 6, 13-23.

Falcioni, F. et al. (1999) Nat. Biotechnol. 17(6), 562-567.

Falk, K. et al. (1991) Nature 351(6324), 290-296.

Fremont, D. H. et al. (1996) Science 272(5264), 1001-1004.

Geitz, R. D. et al. (1995) Yeast 11, 355-360.

Goldstein, J. S. et al. (1998) J. Immunol. 160(7), 3180-3187.

Gregori, S. et al. (2000) Int. Immunol. 12(4), 493-503.

Hammer, J. et al. (1992) J. Exp. Med. 176(4), 1007-1013.

Hammer, J. et al (1993) Cell 74(1), 197-203.

Harrison, L. C. et al. (1997) J. Exp. Med. 185(6), 1013-1021.

Howard, M. C. et al. (1999) Immunol. Today 20(4), 161-165.

Ignatowicz, L. et al. (1995) J. Immunol. 154(8), 3852-3862.

Ishioka, G. Y. et al. (1994) J. Immunol. 152(9), 43104319.

Kieke et al. (1997) Proc. Natl. Acad. Sci. USA 96,5651-5656 Kozono, H. et al. (1994) Nature 369, 151-154.

Kranz, D. M. et al. (1984) Proc. Natl. Acad. Sci. USA 81, 7922-7926.

Lamont, A. G. et al. (1990) J. Immunol. 144, 2493.

Le Doussal, J. et al. (2000) J. Immunol. Methods 241(1-2), 147-58.

Lepley, D. M. et al. (1997) Transplantation 63(5), 765-774.

Madden, D. R. (1995) Annu. Rev. Immunol. 13, 587-622.

Mescher, M. F. et al. (1983) Methods Enzymol. 92, 86-109.

Milik, M. et al. (1998) Nat. Biotechnol. 16(8), 753-756.

Mottez, E. et al. (1995) J. Exp. Med. 181(2), 493-502.

Onda, T. et al. (1995) Mol. Immunol. 32(17-18), 1387-1397.

Rammensee, H. G. et al. (1995) Immunogenetics 41(4), 178-228.

Raymond, C. K. et al. (1999) Biotechniques 26(1), 134-138, 140-141.

Reay, P. A. et al. (1992) EMBO J. 11(8), 2829-2839.

Ribaudo, R. K., and Margulies, D. H. (1995) J. Immunol. 155, 3481-3493.

Ridgway, W. M. et al. (1999) Science 284(5415), 749-751.

Rothbard, J. B. (1991) Annual Review of Immunology 9, 527-565.

Rudensky, A. et al. (1992) Nature 359(6394), 429-431.

Sakita, I. Et al. (1996) J. Immunol. Methods 192(1-2), 105-115.

Schmitt, L. et al. (1999) J. Mol. Biol. 286(1), 207-218.

Scott, C. A. et al. (1998) [published erratum appears in Immunity 1998 Apr; 8(4):531] Immunity 8(3), 319-329.

Sette, A. et al. (1994) Annual Review of Immunology 12, 413-431.

Shields, M. J. et al. (1998) J. Immunol. 160(5), 2297-2307.

Shields, M. J. et al. (1999) Mol. Immunol. 36(9), 561-573.

Shirai, M. et al. (1997) J. Immunol. 158(7), 3181-3188.

Shusta, E. V. et al. (1999) J. Mol. Biol., 292, 949-956.

Smith, J. D. et al. (1995) Mol. Immunol. 32(7), 531-540.

Stern, L. et al. (1994) Nature 368, 215-221.

Stern, L. J., and Wiley, D. C. (1992) Cell 68(3), 465-477.

Udaka, K. et al. (1996) J. Immunol. 157, 670-678.

Uger, R. A. et al. (1999) J. Immunol. 162(10), 6024-6028.

Van der Burg, S. H. et al. (1996) J. Immunol. 156, 3308-3314.

Wucherpfennig, K. W. et al. (1995) Proc. Natl. Acad. Sci. USA 92(25), 11935-11939.

Zhu, X. et al. (1997) Eur. J. Immunol. 27(8), 1933-1941.

What is claimed is:

1. A mutagenized combinatorial library of Major Histocompatibility Complex (MHC) Class I chimeric proteins displayed on the surfaces of recombinant yeast cells, wherein the mutagenized combinatorial library comprises at least one member MHC Class I chimeric protein which is improved in conformational stability or in specific target binding as compared with a comparison MHC Class I chimeric protein which has not been mutagenized.

2. The mutagenized combinatorial library of claim 1 wherein the MHC Class I chimeric protein comprises a portion mediating binding to the surfaces of the recombinant yeast cells and a portion which comprises a specific target binding region of a MHC Class I protein.

3. The mutagenized combinatorial library of claim 2 wherein the portion mediated binding to the surfaces of the recombinant yeast cells is a mating adhesion receptor portion.

4. The mutagenized combinatorial library of claim 3 wherein the mating adhesion receptor portion is an AGA2 portion.

5. The mutagenized combinatorial library of any of claims 2 to 4 wherein the chimeric protein further comprises a portion characterized by an amino acid sequence of a peptide which binds to the binding region of the MHC Class I chimeric protein.

6. The mutagenized combinatorial library of any of claims 2 to 5 wherein the chimeric protein further comprises a portion derived from a c-myc protein and which mediates binding to a c-myc specific antibody.

7. The mutagenized combinatorial library of any of claims 2 to 6 wherein the binding region of the MHC Class I chimeric protein specifically binds a specific target selected from the group consisting of a neoplastic cell, a virus-infected cell, a fungus-infected cell, a parasite-infected cell and a bacterium-infected cell.

8. The mutagenized combinatorial library of claim 8 wherein the peptide binding region specifically binds a peptide having the amino acid sequence given in SEQ ID NO:19, SEQ ID NO:22 or SEQ ID NO:24.

9. The mutagenized combinatorial library of claim 8 wherein said chimeric protein comprises an amino acid sequence as given in SEQ ID NO:17.

10. An isolated mutant MHC Class I chimeric protein, wherein said protein comprises a portion mediating binding to the surfaces of the recombinant yeast cells and a portion which comprises a peptide binding region of a MHC Class I protein and wherein said chimeric protein is improved in stability as compared with an MHC Class I chimeric protein which is not a mutant chimeric protein.

11. The isolated mutant MHC Class I chimeric protein of claim 10 wherein the chimeric protein further comprises a portion comprising an amino acid sequence of a peptide which binds to the peptide binding region of the MHC Class I protein.

12. The isolated mutant MHC Class I chimeric protein of claim 10 wherein a peptide which binds to the peptide binding region of the MHC Class I protein is associated with a neoplastic or infectious disease.

13. The isolated mutant MHC Class I chimeric protein of claim 10 wherein the peptide binding region specifically binds a peptide having the amino acid sequence given in SEQ ID NO:19, SEQ ID NO:22 or SEQ ID NO:24.

14. The isolated mutant MHC Class I chimeric protein of claim 11 wherein said chimeric protein further comprises a detectable label.

15. The isolated mutant MHC Class I chimeric protein of claim 15 wherein the detectable label is a fluorescent moiety, a chromophore, a radionuclide, a chemiluminescent agent, a magnetic particle, an enzyme, a cofactor, a substrate or a toxin.

16. A method for detection of a lymphocyte having a T cell receptor protein in a biological sample, said method comprising the steps of contacting the sample with an isolated mutant chimeric protein of claim 14, wherein said chimeric protein is complexed to the peptide or wherein the chimeric protein and peptide are covalently bound, wherein said chimeric protein comprises a binding region which specifically binds said T cell receptor protein under conditions which allow the binding of the T cell receptor protein to the chimeric protein, and detecting the chimeric protein bound to the T cell receptor protein.

17. The method of claim 16 wherein the T lymphocyte is specific for a neoplastic cell, a tumor cell, a virus-infected cell, a protozoan-infected cell, a bacterium-infected cell or a fungus-infected cell.

18. The method of claim 16 or 17 wherein the biological sample is cells, a tissue sample, biopsy material or bodily fluids.

19. A method for activating or enhancing an immune response to an abnormal cell selected from the group consisting of a neoplastic cell, a tumor cell, a virus-infected cell, a parasite-infected cell, a fungus infected cell or a protozoan infected cell in a human or animal, said method comprising the step of administering to the patient a therapeutically effective amount of an isolated mutant MHC Class I chimeric protein or a mutant MHC Class I chimeric protein/peptide complex which is improved in conformational stability or improved in binding to T lymphocyte as compared with the MHC Class I chimeric protein which is not mutant, whereby the immune response in the human or animal is activated or enhanced.

20. The method of claim 19 wherein the administering is by intravenous, intramuscular, intradermal, subcutaneous or intraperitoneal administration.

21. The method of claim 20 wherein said isolated mutant protein has a portion comprising an amino acid sequence as given in SEQ ID NO:17.

22. The method of claim 21 wherein said mutant protein binds a peptide comprising an amino acid sequence as given in SEQ ID NO:19, SEQ ID NO:22 or SEQ ID NO:24.

23. A combinatorial library of peptides anchored to the surface of yeast cells, each cell displaying a peptide of a unique sequence anchored to its surface, wherein the peptide is anchored to the surface of the yeast cell by a mating factor sequence.

24. The combinatorial library of claim 23 wherein the mating factor sequence is an AGA2 sequence.

25. A method for identifying a peptide which binds specifically to an MHC protein comprising the step of contacting a detectable MHC protein with the combinatorial library of claim 24 or 25 under conditions which allow binding of the protein and the peptide, and detecting peptide bound to the MHC protein.
